(19) United States
(12) Patent Application Publication
(10) Pub. No.: US 2007/0288173 A1
(43) Pub. Date: Dec. 13, 2007

(54) Computational methods and systems to reinforce a humoral immune response

(75) Inventors: Mahalaxmi Gita Bangera, Renton, WA (US); Muriel Y. Ishikawa, Livermore, CA (US); Edward K.Y. Jung, Bellevue, WA (US); Nathan P. Myhrvold, Medina, WA (US); Elizabeth A. Sweeney, Seattle, WA (US); Richa Wilson, San Francisco, CA (US); Lowell L. Wood JR., Bellevue, WA (US)

Correspondence Address: SEARETE LLC;CLARENCE T. TEGREENE, 1756 - 114TH AVE., S.E., SUITE 110, BELLEVUE, WA, 98004, US

(73) Assignee: Searete LLC, a Limited liability corporation of the State of Delware

(21) Appl. No.: 11/728,950

(22) Filed: Mar. 26, 2007

Related U.S. Application Data
(63) Continuation-in-part of application No. 10/925,902, filed on Aug. 24, 2004.
(63) Continuation-in-part of application No. 10/925,904, filed on Aug. 24, 2004.
(63) Continuation-in-part of application No. 10/925,905, filed on Aug. 24, 2004.
(63) Continuation-in-part of application No. 10/926,753, filed on Aug. 25, 2004.
(63) Continuation-in-part of application No. 11/001,259, filed on Dec. 1, 2004.
(63) Continuation-in-part of application No. 11/004,419, filed on Dec. 3, 2004.
(63) Continuation-in-part of application No. 11/004,446, filed on Dec. 3, 2004.
(63) Continuation-in-part of application No. 11/044,656, filed on Jan. 26, 2005.
(63) Continuation-in-part of application No. 11/046,658, filed on Jan. 28, 2005.
(63) Continuation-in-part of application No. 11/131,155, filed on May 16, 2005.
(63) Continuation-in-part of application No. 11/213,325, filed on Aug. 26, 2005.

(51) Int. Cl. G01N 33/48

(52) U.S. Cl. 702/19

(57) ABSTRACT

The present application relates, in general, to a system and/or method for detection and/or treatment.

### CROSS-REFERENCE TO RELATED APPLICATIONS

The present application is related to and claims the benefit of the earliest available effective filing date(s) from the following listed application(s) (the “Related Applications”) (e.g., claims earliest available priority dates for other than provisional patent applications or claims benefits under 35 USC § 119(e) for provisional patent applications, for any and all parent, grandparent, great-grandparent, etc. applications of the Related Application(s)).

### RELATED APPLICATIONS

1. For purposes of the USPTO extra-statutory requirements, the present application constitutes a continuation in part of currently co-pending United States patent application entitled A SYSTEM AND METHOD RELATED TO ENHANCING AN IMMUNE SYSTEM naming MURIEL Y. ISHIKAWA, EDWARD K. Y. JUNG, NATHAN P. MYHRVOLD, RICHA WILSON, AND LOWELL L. WOOD, JR. as inventors, filed 24 Aug., 2004 having U.S. Ser. No. 10/925,902.

2. For purposes of the USPTO extra-statutory requirements, the present application constitutes a continuation-in-part of currently co-pending United States parent application entitled A SYSTEM AND METHOD RELATED TO IMPROVING AN IMMUNE SYSTEM naming MURIEL Y. ISHIKAWA, EDWARD K. Y. JUNG, NATHAN P. MYHRVOLD, RICHA WILSON, and LOWELL L. WOOD, JR. as inventors, filed 24 Aug., 2004 having U.S. Ser. No. 10/925,904.

3. For purposes of the USPTO extra-statutory requirements, the present application constitutes a continuation in part of currently co-pending United States patent application entitled A SYSTEM AND METHOD RELATED TO AUGMENTING AN IMMUNE SYSTEM naming MURIEL Y. ISHIKAWA, EDWARD K. Y. JUNG, NATHAN P. MYHRVOLD, RICHA WILSON, AND LOWELL L. WOOD, JR. as inventors, filed 24 Aug., 2004 having U.S. Ser. No. 10/925,905.

4. For purposes of the USPTO extra-statutory requirements, the present application constitutes a continuation in part of currently co-pending United States patent application entitled A SYSTEM AND METHOD FOR HEIGHTENING AN IMMUNE RESPONSE naming MURIEL Y. ISHIKAWA, EDWARD K. Y. JUNG, NATHAN P. MYHRVOLD, RICHA WILSON, AND LOWELL L. WOOD, JR. as inventors, filed 25 Aug., 2004 having U.S. Ser. No. 10/926,753.

6. For purposes of the USPTO extra-statutory requirements, the present application constitutes a continuation in part of currently co-pending United States patent application entitled A SYSTEM AND METHOD FOR MODULATING A HUMORAL IMMUNE RESPONSE naming MURIEL Y. ISHIKAWA, EDWARD K. Y. JUNG, NATHAN P. MYHRVOLD, RICHA WILSON, AND LOWELL L. WOOD, JR. as inventors, filed 01 Dec., 2004 having U.S. Ser. No. 11/001,259.

7. For purposes of the USPTO extra-statutory requirements, the present application constitutes a continuation in part of currently co-pending United States patent application entitled A SYSTEM AND METHOD FOR HEIGHTENING A HUMORAL IMMUNE RESPONSE naming MURIEL Y. ISHIKAWA, EDWARD K. Y. JUNG, NATHAN P. MYHRVOLD, RICHA WILSON, AND LOWELL L. WOOD, JR. as inventors, filed 03 Dec., 2004 having U.S. Ser. No. 11,004,419.

8. For purposes of the USPTO extra-statutory requirements, the present application constitutes a continuation in part of currently co-pending United States patent application entitled A SYSTEM AND METHOD FOR AUGMENTING A HUMORAL IMMUNE RESPONSE naming MURIEL Y. ISHIKAWA, EDWARD K. Y. JUNG, NATHAN P. MYHRVOLD, RICHA WILSON, AND LOWELL L. WOOD, JR. as inventors, filed 3 Dec. 2004 having U.S. Ser. No. 11/004,446.

9. For purposes of the USPTO extra-statutory requirements, the present application constitutes a continuation in part of currently co-pending United States patent application entitled A SYSTEM AND METHOD FOR IMPROVING A HUMORAL IMMUNE RESPONSE naming MURIEL Y. ISHIKAWA, EDWARD K. Y. JUNG, NATHAN P. MYHRVOLD, RICHA WILSON, AND LOWELL L. WOOD, JR. as inventors, filed 26 Jan., 2005 having U.S. Ser. No. 11/044,656.

10. For purposes of the USPTO extra-statutory requirements, the present application constitutes a continuation in part of currently co-pending United States patent application entitled A SYSTEM AND METHOD FOR MAGNIFYING A HUMORAL IMMUNE RESPONSE naming MURIEL Y. ISHIKAWA, EDWARD K. Y. JUNG, NATHAN P. MYHRVOLD, RICHA WILSON, AND LOWELL L. WOOD, JR. as inventors, filed 28 Jan., 2005 having U.S. Ser. No. 11/046,658.

11. For purposes of the USPTO extra-statutory requirements, the present application constitutes a continuation in part of currently co-pending United States patent application entitled A SYSTEM AND METHOD FOR MAGNIFYING A HUMORAL IMMUNE RESPONSE naming MURIEL Y. ISHIKAWA, EDWARD K. Y. JUNG, NATHAN P. MYHRVOLD, RICHA WILSON, AND LOWELL L. WOOD, JR. as inventors, filed 16 May, 2005 having U.S. Ser. No. 11/131,155.

12. For purposes of the USPTO extra-statutory requirements, the present application constitutes a continuation in part of currently co-pending United States patent application entitled A SYSTEM AND METHOD FOR MODULATING A CELL MEDIATED IMMUNE RESPONSE naming MURIEL Y. ISHIKAWA, EDWARD K. Y. JUNG, NATHAN P. MYHRVOLD, RICHA WILSON, AND LOWELL L. WOOD, JR. as inventors, filed 26 Aug., 2005 having U.S. Ser. No. 11/213,325.

13. For purposes of the USPTO extra-statutory requirements, the present application constitutes a continuation in part of currently co-pending United States patent application entitled COMPUTATIONAL SYSTEMS AND METHODS RELATING TO AMELIORATING AN IMMUNE SYSTEM naming MAHALAXMI GITA BANGERA, MURIEL Y. ISHIKAWA, EDWARD K. Y. JUNG, NATHAN P. MYHRVOLD, ELIZABETH A. SWEENEY, RICHA WILSON, AND LOWELL L. WOOD, JR. as inventors, filed 14 Mar., 2007 having U.S. Ser. No. ______ [To Be Assigned by the USPTO].

14. For purposes of the USPTO extra-statutory requirements, the present application constitutes a continuation in part of currently co-pending United States patent application entitled COMPUTATIONAL SYSTEMS AND METHODS RELATING TO FORTIFYING AN IMMUNE SYSTEM naming MAHALAXMI GITA BANGERA, MURIEL Y. ISHIKAWA, EDWARD K. Y. JUNG, NATHAN P. MYHRVOLD, ELIZABETH A. SWEENEY, RICHA WILSON, AND LOWELL L. WOOD, JR. as inventors, filed 14 Mar., 2007 having U.S. Ser. No. ______ [To Be Assigned by the USPTO].

15. For purposes of the USPTO extra-statutory requirements, the present application constitutes a continuation in part of currently co-pending United States patent application entitled COMPUTATIONAL METHODS AND SYSTEMS TO ADJUST A HUMORAL IMMUNE RESPONSE naming MAHALAXMI GITA BANGERA, MURIEL Y. ISHIKAWA, EDWARD K. Y. JUNG, NATHAN P. MYHRVOLD, ELIZABETH A. SWEENEY, RICHA WILSON, AND LOWELL L. WOOD, JR. as inventors, filed contemporaneously herewith having U.S. Ser. No. ______ [To Be Assigned by the USPTO].

16. For purposes of the USPTO extra-statutory requirements, the present application constitutes a continuation in part of currently co-pending United States patent application entitled COMPUTATIONAL METHODS AND SYSTEMS TO BOLSTER AN IMMUNE RESPONSE naming MAHALAXMI GITA BANGERA, MURIEL Y. ISHIKAWA, EDWARD K. Y. JUNG, NATHAN P. MYHRVOLD, ELIZABETH A. SWEENEY, RICHA WILSON, AND LOWELL L. WOOD, JR. as inventors, filed contemporaneously herewith having U.S. Ser. No. ______ [To Be Assigned by the USPTO].

The United States Patent Office (USPTO) has published a notice to the effect that the USPTO's computer programs require that patent applicants reference both a serial number and indicate whether an application is a continuation or continuation-in-part. Stephen G. Kunin, Benefit of Prior-Filed Application, USPTO Official Gazette Mar. 18, 2003, available at http://www.uspto.gov/web/offices/com/sol/og/2003/week 1/patbene.htm. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant is designating the present application as a continuation-in-part of its parent applications as set forth above, but expressly points out that such designations are not to be construed in any way as any type of commentary and/or admission as to whether or not the present application contains any new matter in addition to the matter of its parent application(s).

All subject matter of the Related Applications and of any and all parent, grandparent, great-grandparent, etc. applications of the Related Applications is incorporated herein by reference to the extent such subject matter is not inconsistent herewith.

### TECHNICAL FIELD

The present application relates, in general, to detection and/or treatment.

### SUMMARY

In one aspect, a system includes but is not limited to: at least one computer program for use with at least one computer system and wherein the computer program includes a plurality of instructions including but not limited to: a first set of instructions for designating one or more computable epitopes of at least one agent; a second set of one or more instructions for predicting one or more changes in the one or more computable epitopes of the at least one agent; and a third set of one or more instructions for aiding the identification of one or more immune response components associated with the one or more computable epitopes of the at least one agent. In addition to the foregoing, other system aspects are described in the claims, drawings, and text forming a part of the present application.

In one aspect, a method includes but is not limited to: designating one or more computable epitopes of at least one agent; predicting one or more changes in the one or more computable epitopes of the at least one agent; and aiding the identification of one or more immune response components associated with the one or more computable epitopes of the at least one agent. In addition to the foregoing, other method aspects are described in the claims, drawings, and text forming a part of the present application.

In one aspect, a method includes but is not limited to: designating one or more computable epitopes of at least one agent associated with a progression of the at least one agent in a host; and designating an immune response component related to treating the host in response to one or more aspects related to the progression of the at least one agent in the host. In addition to the foregoing, other method aspects are described in the claims, drawings, and text forming a part of the present application.

In one aspect, a system includes but is not limited to: circuitry for designating one or more computable epitopes of at least one agent; circuitry for predicting one or more changes in one or more computable epitopes of the at least one agent; and circuitry for aiding the identification of one or more immune response components associated with the one or more computable epitopes of the at least one agent.

In one aspect, a method includes but is not limited to: identifying an association of at least a computable portion of one or more agents with at least a part of an immune response; projecting a pattern of one or more changes relating to the at least a computable portion of the one or more agents; and selecting one or more immune response components in response to the projecting. In addition to the foregoing, other method aspects are described in the claims, drawings, and text forming a part of the present application.

In one aspect, a system includes but is not limited to: circuitry for identifying an association of at least a computable portion of one or more agents with at least a part of an immune response; circuitry for projecting a pattern of one or more changes relating to the at least a computable portion of the one or more agents; and circuitry for selecting one or more immune response components responsive to said circuitry for projecting. In addition to the foregoing, other system aspects are described in the claims, drawings, and text forming a part of the present application.

In one aspect, a method includes but is not limited to: identifying an association of at least one computable epitope of one or more agents with at least a part of an immune response; projecting a pattern of one or more changes relating to the at least one computable epitope of the one or more agents; and selecting one or more immune response components in response to the projecting In addition to the foregoing, other method aspects are described in the claims, drawings, and text forming a part of the present application.

In one aspect, a system includes but is not limited to: circuitry for identifying an association of at least one computable epitope of one or more agents with at least a part of an immune response; circuitry for projecting a pattern of one or more changes relating to the at least one computable epitope of the one or more agents; and circuitry for selecting one or more immune response components in response to the projecting. In addition to the foregoing, other system aspects are described in the claims, drawings, and text forming a part of the present application.

In one aspect, a method includes but is not limited to: accepting an input of one or more agents; and identifying an association of at least one computable portion of one or more agents with at least a part of an immune response related to suppressing the one or more agents. In addition to the foregoing, other method aspects are described in the claims, drawings, and text forming a part of the present application.

In one aspect, a system includes but is not limited to: circuitry for accepting an input of one or more agents; and circuitry for identifying an association of at least one computable portion of one or more agents with at least a part of an immune response related to suppressing the one or more agents. In addition to the foregoing, other system aspects are described in the claims, drawings, and text forming a part of the present application.

In one aspect, a method includes but is not limited to: identifying an association of at least one antigen of one or more agents with at least a part of an immune response; projecting a pattern of one or more changes relating to at least one antigen of the one or more agents; and selecting one or more immune response components in response to the projecting. In addition to the foregoing, other method aspects are described in the claims, drawings, and text forming a part of the present application.

In one aspect, a system includes but is not limited to: circuitry for identifying an association of at least one antigen of one or more agents with at least a part of an immune response; circuitry for projecting a pattern of one or more changes relating to the at least one antigen of the one or more agents; and circuitry for selecting one or more immune response components responsive to said circuitry for projecting. In addition to the foregoing, other system aspects are described in the claims, drawings, and text forming a part of the present application.

In one aspect, a method includes but is not limited to: identifying an association of at least one epitope of one or more agents with at least a part of an immune response; projecting a pattern of one or more changes relating to the at least one epitope of the one or more agents; and selecting one or more immune response components in response to said projecting. In addition to the foregoing, other method aspects are described in the claims, drawings, and text forming a part of the present application.

In one aspect, a system includes but is not limited to: circuitry for associating at least one epitope of one or more agents with at least a part of an immune response; circuitry for projecting a pattern of one or more changes relating to the at least one epitope of the one or more agents; and circuitry for selecting one or more immune response components responsive to said circuitry for projecting. In addition to the foregoing, other system aspects are described in the claims, drawings, and text forming a part of the present application.

In one aspect, a method includes but is not limited to: identifying an association of at least one computable antigen of one or more agents with at least a part of an immune response; projecting a pattern of one or more changes relating to the at least one computable antigen of the one or more agents; and selecting one or more immune response components in response to the projecting. In addition to the foregoing, other method aspects are described in the claims, drawings, and text forming a part of the present application.

In one aspect, a system includes but is not limited to: circuitry for associating at least one computable antigen of one or more agents with at least a part of an immune response; circuitry for projecting a pattern of one or more changes relating to the at least one computable antigen of the one or more agents; and circuitry for selecting one or more immune response components responsive to said circuitry for projecting. In addition to the foregoing, other system aspects are described in the claims, drawings, and text forming a part of the present application.

In one or more various aspects, related systems include but are not limited to circuitry and/or programming for effecting the herein-referenced method aspects; the circuitry and/or programming can be virtually any combination of hardware, software, and/or firmware configured to effect the herein-referenced method aspects depending upon the design choices of the system designer.

In addition to the foregoing, various other method and or system aspects are set forth and described in the text (e.g., claims and/or detailed description) and/or drawings of the present application.

The foregoing is a summary and thus contains, by necessity; simplifications, generalizations and omissions of detail; consequently, those skilled in the art will appreciate that the summary is illustrative only and is not intended to be in any way limiting. Other aspects, inventive features, and advantages of the devices and/or processes described herein, as defined solely by the claims, will become apparent in the non-limiting detailed description set forth herein.

### BRIEF DESCRIPTION OF THE FIGURES

FIG. 1 depicts some aspects of a system that may serve as an illustrative environment of and/or for subject matter technologies.

FIG. 2 depicts a partial view of a system that may serve as an illustrative environment of and/or for subject matter technologies.

FIG. 3 depicts a partial view of a system that may serve as an illustrative environment of and/or for subject matter technologies.

FIG. 4 depicts a diagrammatic view of one aspect of an exemplary interaction of an immune response component, for example, an antibody interacting with an epitope displayed by an agent.

FIG. 5 depicts a diagrammatic view of one aspect of a method of enhancing an immune response.

FIG. 6 depicts some aspects of antigen-antibody interactions showing the occurrence of mutational changes in a selected epitope and corresponding changes in complementary antibodies.

FIG. 7 illustrates one aspect of mutational changes in an epitope displayed by an agent.

FIG. 8 depicts a high-level logic flowchart of a process.

FIG. 9 depicts a high-level logic flowchart depicting alternate implementations of the high-level logic flowchart of FIG. 8.

FIG. 10 depicts a high-level logic flowchart depicting alternate implementations of the high-level logic flowchart of FIG. 8.

FIG. 11 depicts a high-level logic flowchart depicting alternate implementations of the high-level logic flowchart of FIG. 8.

FIG. 12 depicts a high-level logic flowchart depicting alternate implementations of the high-level logic flowchart of FIG. 8.

FIG. 13 depicts a high-level logic flowchart depicting alternate implementations of the high-level logic flowchart of FIG. 8.

FIG. 14 depicts a high-level logic flowchart depicting alternate implementations of the high-level logic flowchart of FIG. 8.

FIG. 15 depicts a high-level logic flowchart depicting alternate implementations of the high-level logic flowchart of FIG. 8.

FIG. 16 depicts a partial view of a system that may serve as an illustrative environment of and/or for subject matter technologies.

FIG. 17 depicts aspects of a system that may serve as an illustrative environment of and/or for subject matter technologies.

FIG. 18 depicts a high-level logic flowchart of a method.

FIG. 19 depicts a high-level logic flowchart depicting alternate implementations of the high-level logic flowchart of FIG. 18.

FIG. 20 depicts a high-level logic flowchart depicting alternate implementations of the high-level logic flowchart of FIG. 18.

The use of the same symbols in different drawings typically indicates similar or identical items.

### DETAILED DESCRIPTION

The present application uses formal outline headings for clarity of presentation. However, it is to be understood that the outline headings are for presentation purposes, and that different types of subject matter may be discussed throughout the application (e.g., device(s)/structure(s) may be described under the process(es)/operations heading(s) and/or process(es)/operations may be discussed under structure(s)/process(es) headings). Hence, the use of the formal outline headings is not intended to be in any way limiting.

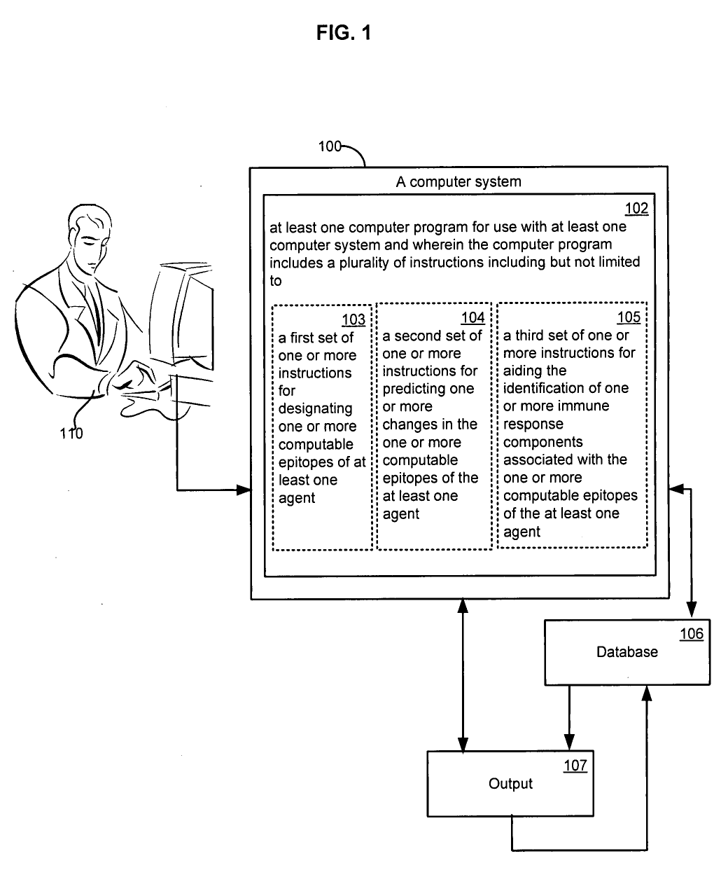

With reference now to FIG. 1, depicted is one aspect of a system that may serve as an illustrative environment of and/or for subject matter technologies, for example, a computer-based method for designating an immune response component for modulating an epitope and/or a computable epitope displayed by an agent. Accordingly, the present application first describes certain specific exemplary systems of FIG. 1; thereafter, the present application illustrates certain specific exemplary structures and processes. Those having skill in the art will appreciate that the specific structures and processes described herein are intended as merely illustrative of their more general counterparts. It will also be appreciated by those of skill in the art that an epitope-antibody, a computable epitope-antibody interaction, and/or an antigen-antibody interaction is an exemplary interaction of an immune response component with an epitope, a computable epitope, and/or an antigen. Therefore, although, the exact nature of the interaction may vary, the overall picture as described herein and/or in other related applications relates to the interaction of an immune response component interacting with the epitope, computable epitope, and/or the antigen. As used herein, the term “epitope” may, if appropriate to context, be used interchangeably with computable epitope, antigen, paratope binding site, antigenic determinant, and/or determinant.

A. Structure(s) and or System(s)

With reference to the figures, and with reference now to FIG. 1, depicted is a partial view of a system that may serve as an illustrative environment of and/or for subject matter technologies. One or more users 110 may use a computer system 100 including at least one computer program for use with at least one computer system and wherein the computer program includes a plurality of instructions 102. The computer program 102 may include one or more sets of instructions, for example, a first set of one or more instructions for designating one or more computable epitopes of at least one agent 103, for example, one or more instructions for designating one or more computable epitopes of at least one agent. The instructions may be such that, when they are loaded to a general-purpose computer or microprocessor programmed to carry out the instructions, they create a new machine, because a general-purpose computer in effect may become a special-purpose computer once it is programmed to perform particular functions pursuant to instructions from program software. That is, the instructions of the software program may electrically change the general-purpose computer by creating electrical paths within the device. These electrical paths, in some implementations, may create a special-purpose machine having circuitry for carrying out the particular program. The computer program 102 may include a second set of one or more instructions 104 that give rise to circuitry for predicting one or more changes in the one or more computable epitopes of the at least one agent, for example, mutations, variations or alternate computable epitopes. The computer program 102 may include a third set of one or more instructions 105 that give rise to circuitry for aiding the identification of one or more immune response components associated with the one or more computable epitopes of the at least one agent, for example, including, but not limited to, a natural and/or a synthetic antibody. The computer program 102 may accept input, for example, from medical personnel, a researcher, wet lab personnel or equipment thereof. A user interface may be coupled to provide access to the computer program 102. In some implementations, the computer program 102 may access at least one database 106 storing information and transmit output 107 to the computer system 100. In one exemplary implementation, a feedback loop is set up between the computer program 102 and the database 106. The output 107 may be fed back into the computer program 102 and/or displayed on the computer system 100. The system may be used as a research tool, as a tool for furthering treatment or the like. This feedback scheme may be useful in an iterative process such as described herein and elsewhere. It will be appreciated by those of skill in the art that the use of the term “set of instructions” may include one or more instructions.

In various aspects, the computer system 100, the computer program 102 and/or the circuitry include predictive algorithms for determining the pattern changes in the computable epitope and the sequence of the computable epitope. In various aspects, the computer system 100, the computer program 102 and/or the circuitry include predictive algorithms for determining the course of a disease influenced by the pattern changes in the computable epitope of the agent. In various aspects, the computer system 100, the computer program 102 and/or the circuitry include computer-based modeling logic for designing and selecting the immune response component for reducing the ability of the agent to establish itself in a host and/or to cause a disease, disorder and/or a condition that may require management. In various aspects, the computer system 100, the computer program 102 and/or the circuitry include logic for integrating with other computer-based systems and incorporating information relevant to selecting an immune response component for modulating the computable epitopes.

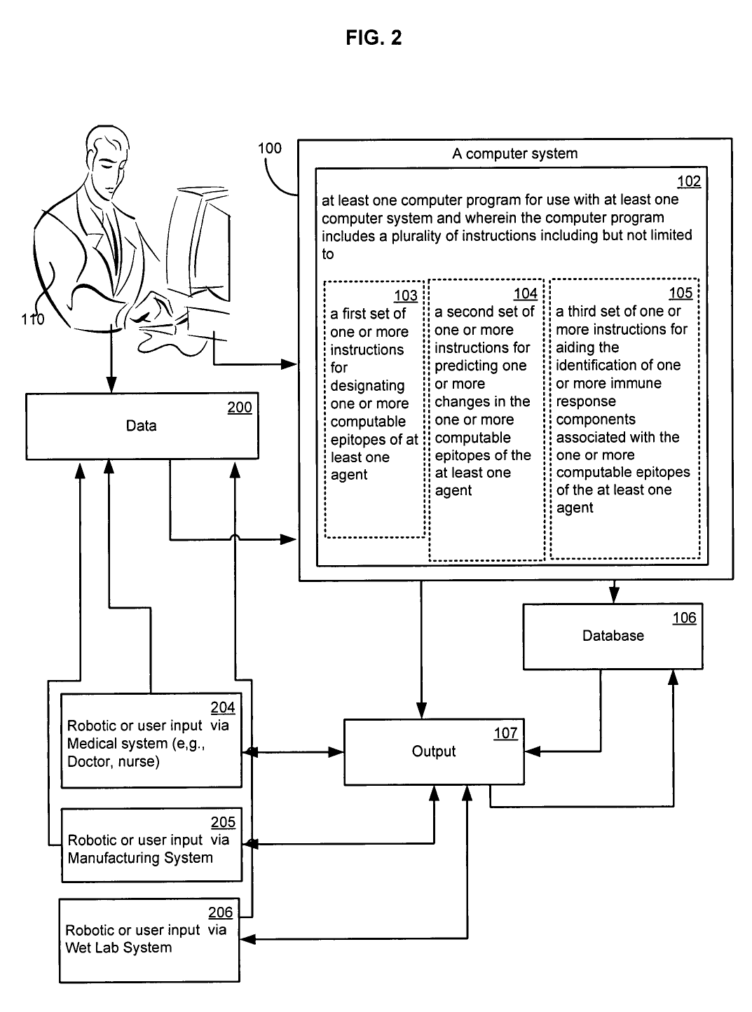

With reference to the figures, and with reference now to FIG. 2, depicted is a partial view of a system that may serve as an illustrative environment of and/or for subject matter technologies. The database 106, data 200, and/or the output 107 may be accessed by various input mechanisms, for example, mechanisms including but not limited to, robotic and/or user input via medical system 204, robotic and/or user input via manufacturing system 205, or robotic and/or user input via wet lab system 206. Access to data 200 may be provided, for example, for further manipulation of the data.

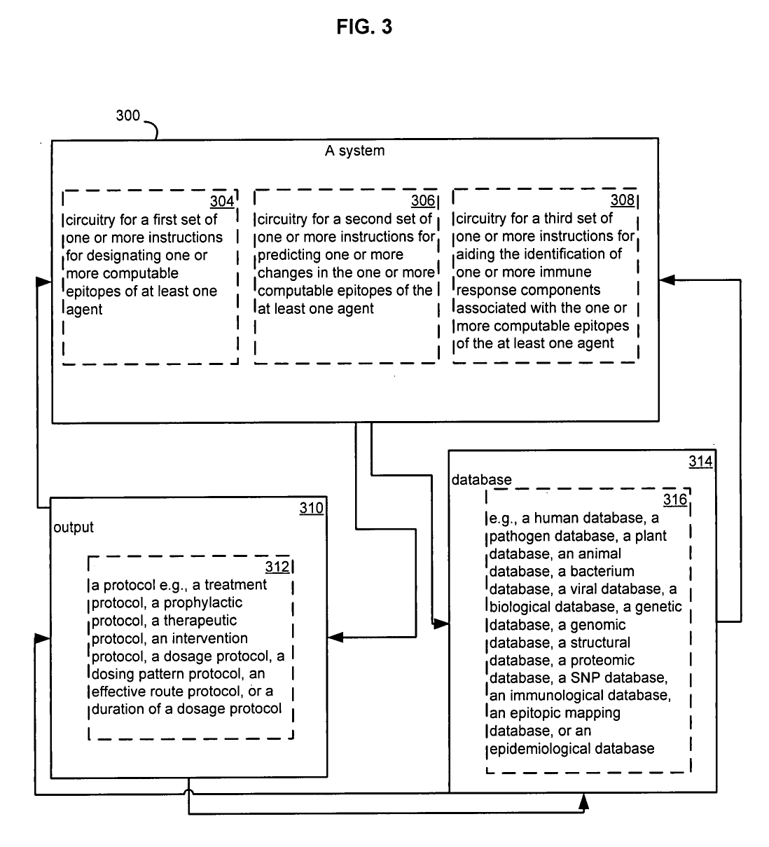

With reference to the figures, and with reference now to FIG. 3, depicted is a partial view of a system that may serve as an illustrative environment of and/or for subject matter technologies. In one aspect, a system 300 may include circuitry and/or components for a first set of one or more instructions for designating one or more computable epitopes of at least one agent 304; circuitry for a second set of one or more instructions for predicting one or more changes in the one or more computable epitopes of the at least one agent 306; and circuitry for a third set of one or more instructions for aiding the identification of one or more immune response components associated with the one or more computable epitopes of the at least one agent 308. Those skilled in the art will recognize that some aspects of the embodiments disclosed herein, in whole or in part, can be equivalently implemented in standard integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and/or firmware would be well within the skill of one of skill in the art in light of this disclosure.

Continuing to refer to FIG. 3, the system 300 may be coupled to at least one database 314 of an identifiable type 316, for example, including, but not limited to, a human database, a host database, a pathogen database, a plant database, an animal database, a bacterium database, a viral database, a fungal database, a protoctist database, a prokaryotic database, an eukaryotic database, a biological database, a genetic database, a genomic database, a structural database, a proteomic database, a SNP database, an immunological database, an epitopic mapping database, and/or an epidemiological database. An output 310 may be displayed, for example, in the form of a protocol 312, for example, including but not limited to a treatment protocol, a prophylactic protocol, a therapeutic protocol, an intervention protocol, a dosage protocol, a dosing pattern protocol, an effective route protocol, and/or a duration of a dosage protocol. In one aspect, the type of output 310 may be selected by the user.

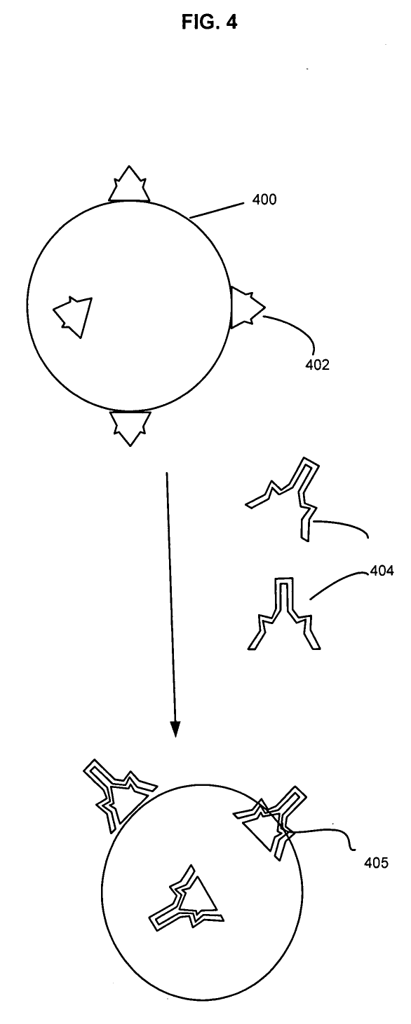

With reference to the figures, and with reference now to FIG. 4, depicted is a diagrammatic view of one aspect of an exemplary interaction of an immune response component, for example, an antibody 404 interacting with an epitope 402 displayed by an agent 400, for example, including but not limited to, in consequence of an interaction involving the agent 400.

The term “immune response component,” as used herein, may include, but is not limited to, at least a part of a macrophage, a neutrophil, a cytotoxic cell, a lymphocyte, a T-lymphocyte, a killer T-lymphocyte, an immune response modulator, a helper T-lymphocyte, an antigen receptor, an antigen-presenting cell, a dendritic cell, a cytotoxic T-lymphocyte, a T-8 lymphocyte, a Cluster of Differentiation (CD) molecule, a CD3 molecule, a CD1 molecule, a B-lymphocyte, an antibody, a recombinant antibody, a genetically engineered antibody, a chimeric antibody, a monospecific antibody, a bispecific antibody, a multispecific antibody, a diabody, a humanized antibody, a human antibody, a heteroantibody, a monoclonal antibody, a polyclonal antibody, a camelized antibody, a deimmunized antibody, an anti-idiotypic antibody, an antibody fragment, a synthetic antibody, and/or any component of the immune system that may bind to an antigen and/or an epitope thereof in a specific and/or a useful manner.

The term “agent,” as used herein, may include, for example, but is not limited to, an organism, a virus, a dependent virus, an associated virus, a bacterium, a yeast, a mold, a fungus, a protoctist, an archaea, a mycoplasma, a phage, a mycobacterium, an ureaplasma, a chlamydia, a rickettsia, a nanobacterium, a prion, an agent responsible for a transmissible spongiform encephalopathy (TSE), a multicellular parasite, a protein, an infectious protein, a polypeptide, a polyribonucleotide, a polydeoxyribonucleotide, a polyglycopeptide, a nucleic acid, an infectious nucleic acid, a polymeric nucleic acid, a metabolic byproduct, a cellular byproduct, and/or a toxin. The term “virus” as used herein, may include, but is not limited to, a provirus, a retrovirus, a virus carrying oncogenes derived from normal cellular genes, and/or an oncovirus, unless context dictates otherwise. The term “agent” may include, but is not limited to, a putative causative agent of a disease, a disorder, a cell and/or component that is deemed, for example, a target for therapy, a target for neutralization, and/or or a cell whose removal lysis or functional degradation may prove beneficial to the host. The term “agent” may also include, but is not limited to, a byproduct or output of a cell that may be neutralized and/or whose removal or functional neutralization may prove beneficial to the host. Furthermore, the term “agent” may include an agent belonging to the same family or group as the agent of primary interest, or an agent exhibiting a common and/or a biological function relative to the agent of primary interest.

The term “antibody”, as used herein, is used in the broadest possible sense and may include, but is not limited to, an antibody, a recombinant antibody, a genetically engineered antibody, a chimeric antibody, a monospecific antibody, a bispecific antibody, a multispecific antibody, a diabody, a humanized antibody, a human antibody, a heteroantibody, a monoclonal antibody, a polyclonal antibody, a camelized antibody, a deimmunized antibody, an anti-idiotypic antibody, and/or an antibody fragment. The term “antibody” may also include, but is not limited to, types of antibodies such as IgA, IgD, IgE, IgG and/or IgM, and/or the subtypes IgG1, IgG2, IgG3, IgG4, IgA1 and/or IgA2. The term “antibody” may also include, but is not limited to, an antibody fragment such as at least a portion of an intact antibody 404, for instance, the antigen binding variable region. Examples of antibody fragments include Fv, Fab, Fab′, F(ab′), F(ab′).sub.2, Fv fragment, diabody, linear antibody, single-chain antibody molecule, multispecific antibody, and/or other antigen binding sequences of an antibody. Additional information may be found in U.S. Pat. No. 5,641,870, U.S. Pat. No. 4,816,567, WO 93/11161, Holliger et al., Diabodies: small bivalent and bispecific antibody fragments, PNAS, 90: 6444-6448 (1993), Zapata et al., Engineering linear F(ab′)2 fragments for efficient production in Escherichia coli and enhanced antiproliferative activity, Protein Eng. 8(10): 1057-1062 (1995), which are incorporated herein by reference. Antibodies may be generated for therapeutic purposes by a variety of known techniques, such as, for example, phage display, and/or transgenic animals and/or organisms.

The term “antibody”, as used herein, may include anti-idiotypic antibodies. Anti-idiotypic antibodies may elicit a stronger immune response compared to the antigen and may be used for enhancing the immune response. Anti-idiotypic antibodies may be rapidly selected, for example, by phage display technology. Additional information may be found in U.S. Patent Application No. 20040143101, to Soltis which is incorporated herein by reference. The term “antibody”, as used herein, may include, but is not limited to, functional derivatives of a monoclonal antibody which include antibody molecules or fragments thereof that have retained a usefully large fraction of the antigenic specificity and/or the functional activity (e.g., of the parent molecule).

The term “heteroantibody,” as used herein, may also include, but is not limited to, two or more antibodies, antibody fragments, antibody derivatives, and/or antibodies with at least one specificity that are linked together. Additional information may be found in U.S. Pat. No. 6,071,517, which is incorporated herein by reference.

The term “chimeric antibody,” as used herein, may include, but is not limited to, antibodies having mouse variable regions joined to human-constant regions. In one aspect, “chimeric antibody” includes antibodies with human framework regions combined with complementarity-determining regions (CDRs) obtained from a mouse and/or rat; those skilled in the art will appreciate that CDRs may be obtained from other sources. Additional information may be found in EPO Publication No 0239400, which is incorporated herein by reference.

The term “humanized antibody,” as used herein, may include, but is not limited to, an antibody having one or more human-derived regions, and/or a chimeric antibody with one or more human-derived regions, also considered the recipient antibody, combined with CDR's from a donor mouse and/or rat immunoglobulin. In one aspect, a humanized antibody may include residues not found in either donor and/or recipient sequences. A humanized antibody may have single and/or multiple specificities. Additional information may be found in U.S. Pat. No. 5,530,101, and U.S. Pat. No. 4,816,567, which are incorporated herein by reference. Information may also be found in, Jones, et al., Replacing the complementarity-determining regions in a human antibody with those from a mouse, Nature, 321:522-525(1986); Riechmann, et al., Reshaping human antibodies for therapy, Nature, 332:323-327 (1988); and Verhoeyen, et al., Reshaping human antibodies: grafting an antilysozyme activity, Science, 239:1534 (1988), which are all incorporated herein by reference.

The term “human antibody,” as used herein may include, but is not limited to, an antibody with variable and constant regions derived from human germline immunoglobulin sequences. The term “human antibody” may include and is not limited to amino acid residues of non-human origin, encoded by non-human germline, such as, for example, residues introduced by site-directed mutations, random mutations, and/or insertions. Methods for producing human antibodies are known in the art and incorporated herein by reference. Additional information may be found in U.S. Pat. No. 4,634,666, which is incorporated herein by reference.

The term “recombinant antibody,” as used herein, may include antibodies formed and/or created by recombinant technology, including, but not limited to, chimeric, human, humanized, hetero antibodies and/or the like.

The term “epitope”, as used herein, may include, but is not limited to, a sequence of at least 3 amino acids, a sequence of at least nine nucleotides, an amino acid, a nucleotide, a carbohydrate, a protein, a lipid, a capsid protein, a polysaccharide, a sugar, a lipopolysaccharide, a glycolipid, a glycoprotein, and/or at least a part of a cell. As used herein, the term “epitope” may, if appropriate to context, be used interchangeably with antigen, paratope binding site, antigenic determinant, and/or determinant. As used herein, the term “determinant” can include an influencing element, determining element, and/or factor, unless context indicates otherwise. In one aspect, the term “epitope” includes, but is not limited to, a peptide-binding site. As used herein, the term “epitope” may include structural and/or functionally similar sequences found in the agent. The term “epitope” includes, but is not limited to, similar sequences observed in orthologs, paralogs, homologs, isofunctional homologs, heterofunctional homologs, heterospecific homologs, and/or pseudogenes of the agent. The epitope 402 may include any portion of the agent. In one aspect, the epitope 402 may include at least a portion of a gene and/or gene-expression product. In another aspect, the epitope may include at least a part of a non-coding region (e.g., of a gene and/or of a genome).

The term “computable epitope” as used herein, includes, but is not limited to, an epitope 402 whose likely future mutable forms may be predicted by using, for example, including, but not limited to, practicable computer-based predictive methodology and/or practicable evolutionary methods and/or practicable probabilistic evolutionary models and/or practicable probabilistic defect models and/or practicable probabilistic mutation models. For example, Smith et al. in their article “Mapping the Antigenic and Genetic Evolution of Influenza-A Virus” on the history of the antigenic evolution of the human influenza-A virus, Science 305, 371 (2004), which is incorporated herein by reference in its entirety, present in this paper's Table 1 and the supporting text thereof a set of patterns of viral coat-protein epitopic evolution which constitutes a basis for predicting one or more patterns of epitopic evolution in this particular agent, which is a well-established threat to human physiological well-being. In one aspect, the computable epitope may be suggested by, for example, including, but not limited to, predictive parallel extrapolations with similar structure, key residues, and/or the presence or absence of known domains. In another aspect, mathematics, statistical analysis and/or biological structural modeling tools may provide the relevant information for designating or identifying the computable epitope. One specific example of a computable epitope is a polypeptide associated with HIV-1, which may be, for example, seven to ten amino acids long. Knowing any starting state of such a peptide (e.g., how the various amino acids are sequenced/arranged), and using current computational techniques, it is practicable to calculate the likely future combinations of the seven to ten amino acids in the polypeptide so as to be able to predict how the epitope will likely appear in future time, as evolution/change occurs in the epitope as biological processes transpire. Indeed, many such evolutionary progressions in the amino acid residue sequences of the viral proteins (e.g., reverse transcriptase and protease) of the several major strains of HIV-1 virus have been reported in the literature, and are used for monitoring the clinical progression of disease in patients. Consequently, in some implementations, technologies described herein computationally predict how the epitope(s) will appear in future mutable forms. This predictive knowledge allows for the designation of at least one immune response component operable for modulating (e.g., reducing and/or eliminating) at least one “future version” of some posited presently existing epitope. As a specific example, one might predict the five or six mostly likely ways in which at least one epitope of a viral protein of a current strain of HIV-1 might appear a few months in the future, and then designate that a person's immune cells be exposed to the chemical structures of the epitopes of such future HIV-1 strains in order to produce an immune response ready, waiting, and keyed to respond to such future HIV-1 strains. Once such antibodies or other immune response components have been produced, amplification or adjuvant techniques may be utilized to produce usefully-large quantities of such antibodies or other immune responses at a time earlier than the elapsing of the three months, and such antibodies administered to a host or a vaccine eliciting such antibodies administered to a host, or cytotoxic responses prepared in the host. Thus, if the HIV-1 does evolve or mutate in at least one of the five or six computationally predicted ways, antibodies or other specific immune responses will be present and waiting to lock onto and negate the HIV-1 virus as it mutates along the predicted paths, thereby effectively precluding its ‘mutational escape’ from iatrogenic or immunological ‘pressure’. Examples listed supra are merely illustrative of methodology that may be used for designating the computable epitope and are not intended to be in any way limiting.

Continuing to refer to FIG. 4, the epitope 402 or parts thereof may be displayed by the agent 400, may be displayed on the surface of the agent 400, extend from the surface of the agent 400, and/or may only be partially accessible to the immune response component. In one aspect, the epitope 402 may be a linear determinant. For example, the sequences may be adjacent to each other. In another aspect, the epitope 402 may be a non-linear determinant, for example, including juxtaposed groups which are non-adjacent ab initio but become adjacent to each other upon protein folding or other assembly. Furthermore, the sequence of the non-linear determinant may be derived by proteasomal processing and/or other mechanisms (e.g., glycosolization, or the superficial ‘decoration’ of proteins with sugars) and the sequence synthetically prepared for presentation to the immune response component.

Continuing to refer to FIG. 4, in one aspect, the immune system launches a humoral response producing antibodies 404 capable of recognizing and/or binding to the epitope 402 followed by the subsequent lysis of the agent 400. Mechanisms by which the antigen 402 elicits an immune response are known in the art and such mechanisms are incorporated herein by reference. In one aspect, the binding of the antibody 404 to the epitope 402 to form an antigen-antibody complex 405 is characterized as a lock-and-key fit. In another aspect, the binding affinity of the antibody 404 for the epitope 402 may vary in time (e.g., in the course of ‘affinity maturation’) or with physiological circumstances. In yet another aspect, the antigen-antibody complex 405 may bind with varying degrees of reversibility. The binding or the detachment of the antigen-antibody complex 405 may be manipulated, for example, by providing a small molecule (possibly solvated) atom, ion, molecule, or compound that promotes the association or disassociation.

In one aspect, the epitope 402 is capable of evoking an immune response. The strength and/or type of the immune response may vary, for example, the epitope 402 may invoke a weak response and/or a medium response as measured by the strength and/or intensity of the immune response, e.g., as may be determined by a resulting peak antibody titer. It is contemplated that, in one instance, the epitope 402 selected for targeting may be one that invokes a weak response in the host, however, it may be selective to the agent 400. In another example, the epitope 402 selected may invoke a weak response in the host, but it may be selected for targeting as it is common to a number of agents deemed to be targets. The herein described implementations are merely exemplary, and should be considered illustrative of like and/or more general implementations within the ambit of those having skill in the art in light of the teachings herein.

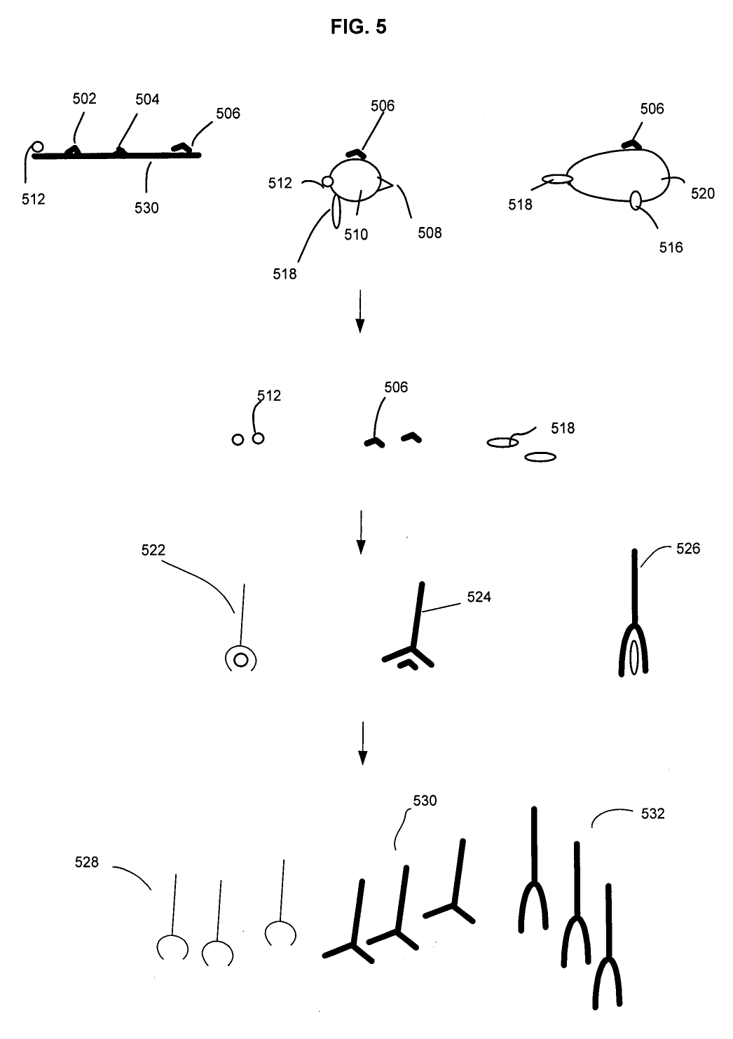

With reference to the figures, and with reference now to FIG. 5, depicted is a diagrammatic view of one aspect of a method of enhancing an immune response. In one aspect, an effective treatment therapy directed towards a disease and/or a disorder may utilize one or more immune response components designed to recognize one or more epitopes common to one or more agents. Such common or shared epitopes may represent an “effective target group” of epitopes. The immune response components designed to seek out and neutralize the common epitopes thereby may be effective against one or more agents. In one aspect, the one or more agents may be subtypes of the agent 400. In this aspect, a set of epitopes may be selected for targeting an agent. In another aspect, the one or more agents may be opportunistic agents capable of aiding or exaggerating an infection or other disease condition formed, caused, or elicited by the agent 400. In yet another aspect, the one or more agents may be agents known to establish a foothold in the host organism prior to or subsequent to an infection or in response to the host's attenuated immune response.

With reference now to FIGS. 4 and 5, in one aspect, a shared epitope 506 is depicted as common to three agents 530, 510 and 520. In another aspect, a second shared epitope 512 is common to two agents 530 and 510. In yet another aspect, a third shared epitope 518 is common to two agents 510 and 520. However, not all epitopes are necessarily shared between agents, for example in FIG. 5 epitope 502 is specific to agent 530. Finding a subset of common epitopes shared amongst one or more agents may be done by statistical analysis, for example, by metaprofiling.

Continuing to refer to FIGS. 4 and 5, in one aspect, one or more agents 530, 510, and 520 depicted may share a subset of common epitopes. The selection of epitopes may depend on many different criteria. For example, the initial selection may be based on selection criteria including, but not limited to, the number of instances of presentation of an epitope 402 by one or more agents, the number of instances of presentation of an epitope 402 by an agent 400, the location of an epitope 402 in or on the agent, the size of an epitope 402, the nature of an epitope 402, the comparative sequence identity and/or homology of an epitope 402 with one or more host sequences, the composition of an epitope 402, and/or putative known or predicted changes in an epitope 402 sequence. The selection of epitopes may also depend on, for example, the type of immune response component desired for treating and/or managing the disease, disorder, and/or condition.

In one aspect, an epitope 402 selected has a probable sequence match with another agent of interest, for example, an opportunistic agent, or a subsequent, prior, or concurrent infection of the host caused by another agent. In another aspect, an epitope 402 selected has a low degree of probable match with the host, for example, in order to decrease possible side effects due to the production and/or eliciting of self- or auto-antibodies. The term “host,” as used herein, may include, but is not limited to, an individual, a person, a patient, and/or virtually any organism requiring management of a disease, disorder, and/or condition. For example, including, but not limited to, an epitope 402 selected may have a 0-70% sequence match at the amino acid level with the host or the agent 400, or a 0-100% sequence match with the agent. Those having skill in the art will recognize that part of that context in relation to the term “host” is that generally what is desired is a practicably close sequence match to the agent (e.g., HIV-1 or influenza-A virus), so that the one or more immune system components in use can attack it and a practicably distant sequence match to the host (e.g., a patient), in order to decrease or render less aggressive or less likely any attack by the immune system components in use on the host. However, it is also to be understood that in some contexts the agent will in fact constitute a part of the host (e.g., when the agent to be eradicated is actually a malfunctioning part of the host, such as in an auto-immune or neoplastic disease), in which case that part of the host to be eradicated will be treated as the “agent,” and that part of the host to be left relatively undisturbed will be treated as the “host.” In another aspect, the epitope 402 selected has a sequence match with the agent, for example, a high degree of sequence match, or a relatively higher degree of sequence match with other agents compared to the host, or a 0-100% sequence match with the agent 400. The term “sequence match,” as used herein, includes sequence matching at the nucleic acid level, at the polysaccharide level, at the protein level, and/or at the polypeptide level. In an embodiment, an epitope 402 selected has a low percent sequence match with the host. In another embodiment, an epitope 402 selected has a high degree of sequence match with other agents.

In molecular biology, the terms “percent sequence identity,” “percent sequence homology,” “percent sequence similarity” or “percent sequence match” are sometimes used interchangeably. In this application these terms are also often used interchangeably, with each other, and with “degree of sequence match,” unless context dictates otherwise.

In another aspect, an epitope 402 selected has a likely and/or a high percent sequence match with other epitopes, for example, including, but not limited to, an epitope 402 having a structural sequence match, a functional sequence match, a similar functional effect, a similar result in an assay and/or a combination. Structural comparison algorithms and/or 3-dimensional protein structure data may be used to determine whether two proteins or presented fragments thereof may have a usefully-high percent structural sequence match. In another example, an epitope 402 may have a functional match and/or share a similar functional effect with epitopes of interest. In this example, an epitope 402 may have a lower percent sequence match but may still exert the same functional effect.

In another example, an epitope 402 and/or other epitopes of interest may have a lower percent sequence match but may share similar activities, for example, enzymatic activity and/or receptor binding activity, e.g., as determined by the use of an assay.

In another aspect, an epitope 402 selected may be an immunological effective determinant; for example, an epitope 402 may be weakly antigenic, but it may evoke an effective immune response deriving from, for example, the nature and/or the type of the immune response component that it induces. In another aspect, an epitope 402 may exert a similar effect on the immune response. For example, an epitope 402 selected may be part of the antigenic structure of an agent unrelated to the disease or disorder in question, however, it may exert a substantially similar effect on the immune system as assessed by, for example, the type, the nature, and/or the time-interval of the immune response.

In one aspect, a sequence match with an entity may be determined by, for example, calculating the percent identity and/or percent similarity between epitopes and/or between an epitope 400 and the epitopic sequences of the host. In one aspect, a sequence match with an entity may be quantified by, for example, calculating the percent identity and/or percent similarity between epitopes and/or between the epitope 400 and the host. In one aspect, the percent identity between two sequences may be calculated by determining a number of substantially similar positions obtained after aligning the sequences and introducing gaps. For example, in one implementation the percent identity between two sequences is treated as equal to (=) {a number of substantially similar positions÷the total number of positions}×100. In this example, the number and length of gaps introduced to obtain optimal net alignment of the sequences to be considered. In another aspect, the percent identity between two sequences at the nucleic acid level may be determined by using a publicly-available software tool such as BLAST, FASTA, BLAST-2, ALIGN and/or DNASTAR software. Similarly, the percent identity between two sequences at the amino acid level may be calculated by using publicly available software tools such as, for example, Peptidecutter, AACompSim, Find Mod, GlycoMod, InterProtScan, DALI and/or tools listed on the ExPasy Server (Expert Protein Analysis System) Proteomics Server at http://www.expasy.org/. In some embodiments, the percent identity at the nucleic acid level and/or at the amino acid level are determined.

In one aspect, string matching algorithms may be used to identify homologous segments, for example, using FASTA, and BLAST. In another aspect, sequence alignment based on fast Fourier transform (FFT) algorithms may be used to rapidly identify homologous segments. In yet another aspect, iterative searches may be used to identify and select homologous segments. Searches may be used not only to identify and select shared epitopes but also to identify epitopes that have the least homology with human sequences. Additional information may be found in Katoh et al., MAFFT: a novel method for rapid multiple sequence alignment based on fast Fourier transform, Nucleic Acids Research, 30(14):3059-66 (2002) which is incorporated herein by reference.

A number of large-scale screening techniques may be used to identify and select the designed antibody, for example, the antibody designed may be selected by using optical fiber array devices capable of screening binding molecules. Additional information may be found in U.S. Patent Application No. 20040132112 to Kimon et al., which is hereby incorporated by reference.

It will be appreciated by those skilled in the art that an epitope 402 selected need not be limited to a matching sequence displayed by an agent 400. In one aspect, a meta-signature and/or a consensus sequence may be derived based on any number of criteria. In one aspect, the meta-signature may be derived by analysis of data from sources such as, for example, antigenic evolution, genetic evolution, antigenic shift, antigenic drift, data from crystal structure, probable match with a host, probable match with other agent strains, and/or strength of the immunogenic response desired. The meta-signature may include new sequences and/or may exclude some sequences. For example, it may include silent mutations, mismatches, a spacer to bypass a hotspot or a highly mutable site, predicted changes in the sequence, and/or may include epitopes from multiple agents thus providing immune-based protection from multiple agents. As another example, the meta-signature may exclude sequences, such as, for example, including, but not limited to, mutable sequences and/or sequences with a high percent sequence match to the host's epitopes. As another example, the meta-signature may exclude sequences, such as, for example, including, but not limited to, mutagenic sequences and/or sequences with a high percent sequence match to a host sequence.

In one aspect, the predicted changes in an epitope 402 may be determined by analysis of past variations observed and/or predicted in an agent 400 (e.g., FIG. 5). Computational analysis can be used to determine regions showing sequence variations and/or hot spots. In one aspect, high speed serial passaging may be performed in silico, computationally mimicking the serial passaging that occurs naturally with production of a new strain of an agent 400. It will be appreciated by those of skill in the art that the hot spots need not be identified by examining an epitope 402, and/or by examining an epitope 402 in context with an agent 400. Information pertaining to hot spots can also be extrapolated by performing sequence analysis of other agents and/or domain analysis of such other agents. For example, in some implementations, an epitope 402 may be part of a domain shared between multiple agents, some of which may lack an epitope 402 of interest. Information pertaining to hot spots identified in the domain of the other agents may be of practical use in determining the meta-signature.

In one aspect, one or more sets and/or subsets of epitopes may be formed. The nature and type of criteria used to form the sets and/or subsets will depend, for example, on the nature and type of an agent 400, the duration of the immune response desired (e.g., short-term immunity, or long-term immunity), the nature of the immune response desired (e.g., weak, moderate, or strong), the population to be protected (e.g., presence and/or currency of varying degrees of prior agent exposure), and the like. The sets and/or subsets so formed may accept input either robotically or from a user (e.g., from a manufacturer of immune response components, from a wet lab, and/or medical or research personnel).

The pattern changes predicted in an epitope 402 may be supplemented, for example, by other methodology, statistical analysis, historical data, and/or other extrapolations of the type utilized by those having skill in the art. The knowledge of these predicted pattern changes represents an asset in the design and/or selection of the immune response components. The predicted pattern changes may be used to determine the progression of the changes in the immune response component required to manage such changes. Inferring pattern changes in an epitope 402 and using the information to modulate the progressing response may aid in managing the response more effectively. For example, the pattern changes may be used to provide a timeline of when the therapy could be changed, what therapy should constitute the change, or the duration of the change. As a more specific example, one reason why HIV-1 virus is able to eventually kill its host is that the virus mutates its antigenic signature-profile significantly faster than the immune system can effectively track and respond to these mutations. In a specific implementation of the subject matter described herein, a sample of HIV-1 is taken from a patient at a point in time and computational biological techniques are used to infer likely mutations of the antigenic signature-profile of the virus at future times. Techniques such as cloning are then utilized to synthesize immune system-activating aspects of the anticipated future HIV-1 strains, and thereafter replicative techniques are utilized to rapidly generate copious amounts of one or more immune system components (e.g., antibodies) that are keyed to the likely future generation of the patient's particular strain and sub-strain(s) of HIV-1. Once cloned, the immune system components are then loaded back to the patient and thus are “present and waiting” for the HIV-1 viral quasispecies components when it mutates into the anticipated new forms and/or attempts to proliferate these forms. If a HIV-1 quasispecies mutates as anticipated, the “preloaded” immune response components may successfully negate the mutated quasi-species components, thereby likely greatly reducing the patient's viral load—and crucially suppressing the likelihood of further mutational evolution since the virion population of mutated forms never becomes substantial. In some implementations, the mutational history of the HIV-1 quasi-species is closely tracked, and once the actual mutational direction has been determined, high-speed (likely, ex vivo) techniques are utilized to generate yet more immune system components capable of effective suppression appropriate of the mutated viral quasispecies, significantly more rapidly than the virus is able to effectively mutate and thus ‘escape’ from the suppressive therapy.

In one aspect, the epitope 402 selected for designating the immune response component may be synthetically made and/or derived from the agent 400. In one embodiment, the epitope 402 selected is derived from an agent 400 extracted from an individual desiring treatment and/or an individual found to be resistant to that agent. In one aspect, the epitope 402 selected for designating the immune response component may include multiple copies of the exact same epitope and/or multiple copies of different epitopes.

In one aspect, the meta-signature includes sequences matching adjacent and/or contiguous sequences. In another aspect, the meta-signature includes non-adjacent sequences. For example, it will be appreciated by those of skill in the art that peptide splicing and/or proteosomal processing of an epitope 402 that occurs naturally may result in the formation of a new epitope, for example, a non-linear epitope. In this example, proteosomal processing may result in the excision of sequences and the transposing and/or juxtaposing of non-contiguous sequences to form the non-linear epitope. Additional information may be found in Hanada, et al., Immune recognition of a human renal cancer antigen through post-translational protein splicing, Nature 427:252 (2004), and Vigneron, et al., An antigenic peptide produced by peptide splicing in the Proteosome, Science 304:587 (2004), hereby incorporated by reference herein in their entirety.

Additionally, it will also be appreciated by those of skill in the art that a meta-signature may include sequences displayed on two different parts of an agent 400. For example, non-adjacent sequences may appear adjacent each other when the protein is folded. In this aspect, a meta-signature may include the non-adjacent sequences for identifying a meta-signature. Furthermore, a meta-signature may include non-adjacent sequences corresponding to a specific conformational state of a protein. Immune response components designed to bind such sequences may be specific to the conformational state of the protein. 3-D and/or crystal structure information may also be used to designate a meta-signature. In one aspect, the meta-signature may include multiple sets of epitopes targeting a predicted pattern change and/or an observed pattern change. For example, multiple sets of epitopes may be designed for vaccination and/or for production of immune response components.

Techniques for epitope mapping are known in the art and herein incorporated by reference. For example, FACS analysis and ELISA may be used to investigate the binding of antibodies to synthetic peptides including at least a portion of the epitope. Epitope-mapping analysis techniques, Scatchard analysis and the like may be used to predict the ability of an antibody 404 to bind to an epitope 402 presented on an agent 400, to determine the binding affinity of an antibody 404 or other immune response component to an epitope 402, and/or to discern a desirable configuration for an antibody 404 or other immune response component.

Continuing to refer to FIG. 5, in one aspect, for example, the sequences of selected epitopes 506, 512, and 518 may be used to design one or more complementary antibodies 524, 522, or 526, or other immune response components, respectively. The sequences of selected epitopes 506, 512, and 518 may be used to form monoclonal antibodies, for example, by cloning or by using human mouse systems. The sequences of selected epitopes 506, 512, and 518 may be amplified using the polymerase chain reaction (PCR) as described in U.S. Pat. Nos. 4,683,195, 4,683,202, and 4,800,159 to Mullis, et al. which are incorporated herein in their entirety. In another aspect, a consensus sequence and/or a meta-signature may be designed and amplified. The relevant sequence(s) may be inserted in an expression vector for producing proteins and the expressed protein(s) subsequently used to produce antibodies specific to the selected epitopes. In one aspect, selected epitopes may be antigenic but may not be directly immunogenic.

Human antibodies may be made, for example, by using a human mouse system such as, for example, the Xenomouse technology of Abgenix, Inc., (available from Abgenix, Inc, now a branch of Amgen, Inc, with headquarters in Fremont, Calif. 94555) and/or the HuMAb Mouse technology of Medarex, Inc., (available from Medarex Inc. currently having corporate headquarters in Princeton, N.J.). In these systems, the host mouse immunoglobulins genes are inactivated and human immunoglobulin genes are inserted in the host. On stimulation with an antigen, such transgenic mice produce fully human antibodies. Subsequently, human monoclonal antibodies can be isolated according to standard hybridoma technology.

Selection of humanized antibodies with higher binding affinities from promising murine antibodies may be performed by using computer modeling software developed by Queen, et al. The antibodies produced by this method include approximately 90% of the pertinent human sequences. The structure of the specific antibody is predicted based on computer modeling and the retaining of key amino acids predicted to be necessary to retain the shape and, therefore, the binding specificity of the complementarity determining regions (CDR's). Thus, key murine amino acids are substituted into the human antibody framework along with murine CDR's. The software may then be used to test the binding affinity of the redesigned antibody with the antigen. Additional information can be found in U.S. Pat. No. 5,693,762 to Queen, et al., which is incorporated herein by reference.

The formation of other antibody fragments, such as, for example, Fv, Fab, F(ab′).sub.2 or Fc may be carried out by, for example, phage antibody generated using the techniques as described in McCafferty et al., Phage antibodies: filamentous phage displaying antibody variable domains, Nature 348:552-554 (1990), and Clackson, et al., Making Antibody Fragments Using Phage Display Libraries, Nature 352:624-628 (1991) and U.S. Pat. No. 5,565,332 to Hoogenboom, et al., which are incorporated herein by reference. Surface plasmon resonance techniques, for instance, may be used to analyze real-time biospecific interactions. Camelized antibodies, deimmunized antibodies and anti-idiotypic antibodies may be selected by techniques known in the art, which are herein incorporated by reference.

In one aspect, the selection of antibodies for modulating the immune response may be based on their function. For example, activating antibodies, blocking antibodies, neutralizing antibodies, and/or inhibitory antibodies may be used to modulate the immune response. Such antibodies may perform one or more functions under the appropriate conditions. In a more specific example, an antibody 404 may be triggered to undergo a conformational change by providing a cofactor and/or by changing the ambient temperature or other ambient conditions, such as overall osmolality or pH or concentration of a particular compound, atom or ion. The conformation change may result in a new function being performed by an antibody 404.

Techniques for purifying antibodies are known in the art and are incorporated herein by reference. The purified complementary antibodies 530, 528, or 532 may be made available for therapeutic and/or prophylactic treatment.

The term “an effective treatment therapy,” as used herein, includes, but is not limited to, the use of immune response components in combination with other antibodies, antibody fragments, and/or in combination with other treatments, including, but not limited to, drugs, vitamins, hormones, medicinal agents, pharmaceutical compositions and/or other therapeutic and/or prophylactic combinations. In another aspect, the immune response component may be used in combination, for example, with a modulator of an immune response and/or a modulator of an antibody. In one aspect, cocktails of immune response components may be administered, for example, by injecting or otherwise effectively inserting by a subcutaneous, nasal, intranasal, intramuscular, intravenous, intraarterial, intrathecal, intracapsular, intraorbital, intracardiac, transdermal, subdermal, intradermal, intraperitoneal, transtracheal, subcuticular, intraarticular, subcapsular, subarachnoidal, intraspinal, epidural, intrasternal, infusion, topical, sublingual, and/or enteric route.

The therapeutic effect of the immune response component may be produced by one or more modes of action. For example, in one aspect, the immune response component may produce a therapeutic effect and/or alleviate the symptoms by targeting specific cells and neutralizing them. In another aspect, the immune response component may bind to and/or block receptors present on an agent 400 and/or may directly and/or indirectly block the binding of molecules, such as, for example, cytokines, and/or growth factors, or modulators or pro- or anti-apoptotic signaling materials to an agent 400. In another aspect, the therapeutic effect of the immune response component is produced by functioning as signaling molecule(s). In this example, the immune response component(s) may induce cross-linking of receptors with subsequent induction of programmed cell death (e.g., apoptosis).

The immune response component may be engineered to include, for example, one or more effector molecules, such as, for example, drugs, small molecules, enzymes, toxins, radionuclides, cytokines, and/or DNA molecules. In this example, the immune response component may serve as a vehicle for targeting and binding an agent 400 and/or delivering the one or more effector molecules. In one aspect, the immune response component may be engineered to include the one or more effector molecules without the natural effector functions of the immune response component. In one aspect, one or more immune response components may be coupled to molecules for promoting immune system components to act to eliminate unwanted cells or other biological entities, such as virus particles. This technique has been described for the treatment of tumors, viral-infected cells, fungi, and bacteria using antibodies. Additional information may be found in U.S. Pat. No. 4,676,980 to Segal, which is incorporated herein by reference.

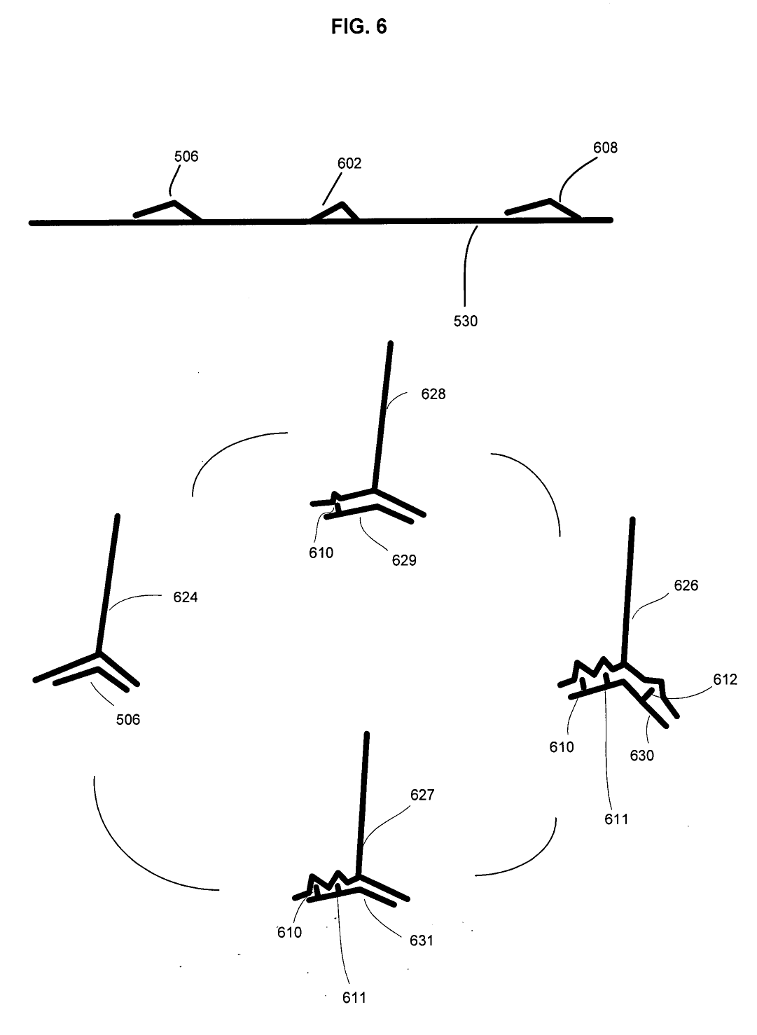

With reference to the figures, and with reference now to FIG. 6, depicted is one aspect of an antigen antibody-interaction showing the occurrence of mutational changes in a selected epitope and corresponding changes in a complementary antibody. A selected epitope 506 may undergo mutational changes. Other epitopes 602 and/or 608 may not be selected, for example, as the mutation rate for these epitopes may be substantially high. These mutations may be random and, therefore, non-predictable, or they may be predictable. For example, a mutation may be substantially more predictable based on the occurrence of “hot spots” or known mutational history. The complementary antibody 624 may bind the selected epitope 506, for example, with a usefully-high affinity. However, a sequence change 610 depicted in a mutated selected epitope 629 may reduce the binding affinity of the complementary antibody 624 or other immune response component. A complementary antibody incorporating the mutation 628 may restore the higher binding affinity, for example, to a usefully-high binding affinity. Similarly, appearance of mutations 611 and 612 may require a new complementary antibody 626 or other immune response component in order to attain a usefully-high binding affinity. Additionally, the appearance of mutations 610 and 611 may require a new complementary antibody 627 or other immune response component. The predictive aspect of the computer system, software and/or circuitry may be used to make mathematical predictions regarding the mutational variations and the treatment components required or likely to be of utility in addressing them. In one aspect, the complementary antibody or other immune response component need not have a high binding affinity. For example, the new antibody 626 or other immune response component may be used to bind and modulate the agents with mutations 610, 611 and/or 612.

With reference to the figures, and with reference now to FIGS. 4, 5, and 6, depicted is one aspect of the antigen-antibody interaction showing the occurrence of mutational changes in the selected epitope 506 and corresponding changes in the complementary antibody 524 or other immune response component. Such mutational changes in the selected epitope 506, for example, may be minor or major in nature. These minor and/or major antigenic variations may render an existing treatment less effective. Thus an effective treatment therapy towards a disease or disorder may include treating the disease or disorder with one or more antibodies designed to anticipate one or more predictable antigenic variations, for example, including, but not limited to, one or more agents or one or more related agents, and/or shared with at least two agents. Furthermore, predicting the course of the minor and/or major antigenic variations of an agent 400 and/or the related agents would also be beneficial in designing or selecting these anticipatory antibodies. Additionally, in some implementations the inclusion of information from SNP databases may be useful in designing antibodies for binding the selected epitope 506.

Minor changes in the epitope 402 which do not always lead to the formation of a new subtype may be caused, for example, by point mutations in the selected epitope 506. In one aspect, the occurrence of point mutations may be localized, for example, to hot spots of the selected epitope 506. The frequency and/or occurrence of such hot spots may be predicted by the computer-based method. Additionally, the method provides for access to databases including, for example, historical compilations of the antigenic variations of the agent 400 and/or of the selected epitope 506, for example, from previous endemics and/or pandemics or the natural evolutionary history of the disease. Such information may be part of an epitope profile for charting the progression of the immune response. A non-exclusive example is provided by a point mutation of the glutamic acid residue at position 92 of the NS1 protein of the influenza-A virus that has been shown to dramatically down-regulate activation of human cytokines. Such information may be useful in designating the meta-signature.

Continuing to refer to FIGS. 4, 5, and 6, depicted is that a mutation 610 in the selected epitope 506 results in a mutated epitope 629. The term “the selected epitope 506” as typically used herein, often constitutes a type of the more general term of presented epitope, unless context indicates otherwise. The generation of the mutated epitope 629 may reduce the binding of the immune response component, for example, the antibody 624. In one aspect, effective binding could be enhanced by generating a new antibody 628 corresponding to the mutated epitope 610. The frequency of minor antigenic variations may be predicted by examining known and/or predicted mutational hot spots. For example, additional mutations 611 and/or 612 may be predicted by a computer-based method and corresponding antibodies 626 and/or 627 respectively may be designed to compensate for such antigenic variations in mutated epitopes 630 and/or 631, respectively. In one aspect, an effective treatment therapy may incorporate this knowledge in the course of providing an effective humoral response towards an agent 400. For example, a cocktail of immune response components may include antibodies 624, 628, 626, and/or 627 for binding to selected epitope 506 and/or its predicted mutated versions. In one aspect, the cocktail of one or more antibodies or other immune response components may be supplemented by additional chemicals, drugs, and/or growth- or replication- or survival-modulating factors. In another aspect, an effective treatment therapy may include varying doses of immune response components, for example, a substantially larger or more prolonged or earlier- or later-administered dosage of 626 relative to 624, 628, and/or 627.

In another aspect, the antibodies or other immune response components with higher binding affinities may be selected. Numerous techniques exist for enhancing the binding affinity of the antibody or other immune response component for an epitope 402. In one aspect, the binding affinity of the antibody or other immune response component for an epitope 402 may be enhanced by constructing phage display libraries from an individual who has been immunized with an epitope 402 either by happenstance or by immunization. The generation and selection of higher affinity antibodies may also be improved, for example, by mimicking somatic hypermutagenesis, complementarity-determining region (CDR) walking mutagenesis, antibody chain shuffling, and/or technologies such as Xenomax technology (available from Abgenix, Inc, now a division of Amgen, Inc, with headquarters in Fremont, Calif. 94555). In one example, antibodies including introduced mutations may be displayed on the surface of filamentous bacteriophage. Processes mimicking the primary and/or secondary immune response may then be used to select the desired antibodies, for example, antibodies displaying a higher binding affinity for the antigen, and/or by evaluating the kinetics of dissociation. For additional information see, Low, et al., Mimicking Somatic Hypermutation: Affinity Maturation Of Antibodies Displayed On Bacteriophage Using A Bacterial Mutator Strain, J. Mol. Biol. 260:359-368 (1996); Hawkins, et al. Selection Of Phage Antibodies By Binding Affinity. Mimicking Affinity Maturation, J. Mol. Biol. 226:889-896 (1992), which are incorporated herein by reference.

As an example, the generation and/or selection of higher affinity antibodies may be carried out by CDR walking mutagenesis, which mimics the tertiary immune selection process. For example, saturation mutagenesis of the CDR's of an antibody 404 may be used to generate one or more libraries of antibody fragments which are displayed on the surface of filamentous bacteriophage followed by the subsequent selection of the relevant antibody using immobilized antigen. Sequential and parallel optimization strategies may be used to then select the higher affinity antibody. For additional information see Yang, et al., CDR Walking Mutagenesis For The Affinity Maturation Of A Potent Human Anti-HIV-1-1 Antibody Into The Picomolar Range, J. Mol. Biol. 254(3):392-403 (1995), which is incorporated herein by reference in its entirety. In another example, site-directed mutagenesis may be used to generate and select higher affinity antibodies, for example, by parsimonious mutagenesis. In this example, a computer-based method is used to identify and screen amino acid residues included in the one or more CDR's of a variable region of an antibody 404 involved in an antigen-antibody binding. Additionally, in some implementations, the number of codons introduced is such that about 50% of the codons in the degenerate position are wildtype. In another example, antibody-chain shuffling may be used to generate and select higher affinity antibodies. These techniques are known in the art and are herein incorporated by reference.

The dosage of the immune response component may vary and in one aspect may depend, for example, on the duration of the treatment, body mass, history and/or characteristic of the disease, health-history, genotype, sex and/or age. Compositions including immune response components may be delivered to an individual for prophylactic and/or therapeutic treatments. In one aspect, an individual having a disease and/or condition is administered a treatment dose to alleviate and/or at least partially cure the condition manifested by the symptoms. In this example, a therapeutically effective dose is administered to the host-patient.

In another aspect, a host's resistance to disease conditions may be enhanced by providing a prophylactically calibrated dose of an antibody 404. A prophylactic dose may be provided to, for example, including, but not limited to, a potential host-individual genetically predisposed to a disease and/or a condition, a potential host (about to be) present in a region where a disease is prevalent, and/or a potential host-individual wishing to enhance that host-individual's immune response.

Optimization of the physico-chemical properties of the immune response component may be improved, for example, by computer-based screening methods. Properties affecting antibody therapeutics may also be improved, such as, for example, stability, antigen binding affinity, and/or solubility. Additional information may be found in U.S. Patent Application No. 20040110226 to Lazar, which is incorporated herein by reference.

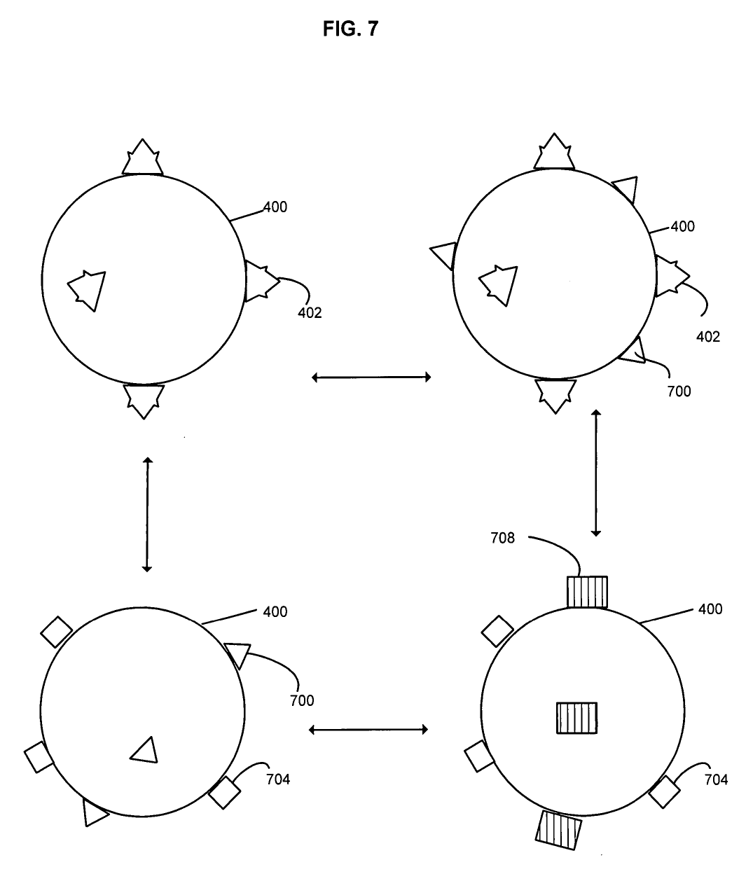

Referring now to FIG. 7, illustrated is one aspect of mutational changes in an epitope displayed by an agent and the corresponding changes in an immune response component, for example, one or more new epitopes 700 and/or 704 may appear on the surface of an agent 400. In one aspect, major changes may occur in the antigenic variants present on the surface of an agent 400, resulting in the formation of a new subtype or sub-strain. The appearance of new epitopes observed, for example, may occur as a result of antigenic shifts, reassortment, reshuffling, rearrangement of segments, and/or swapping of segments, and often marks the appearance of a new virulent and/or pathogenic (sub-) strain of an agent 400. In one instance, the prediction of the new epitopes may mark the emergence of a new (sub-) strain, a new subtype, and/or the reemergence of an older (sub-) strain. In this instance, natural and/or artificial immune protection in an individual alone may not provide adequate and/or effective protection against initial infection or infection-progression. Immune protection and/or humoral protection may be supplemented with, for example, drugs, chemicals or small molecules capable of enhancing, supplanting or favorably interacting with the effects of the pertinent immune response components.

Generally, when major epitopic changes do occur, a larger section of the exposed host population succumbs to the infection, sometimes leading to an epidemic or a pandemic. This problem may be alleviated in part, for example, by predicting the appearance of new (sub-) strains and/or subtypes of an agent as a result of the appearance of new epitopes and/or the disappearance of existing epitopes. In one aspect, for example, including, but not limited to, the prediction of the new epitopes, attention may be directed towards a subset of genes, for example, those important for overall Darwinian fitness, replication and/or infectivity of an agent 400. For example, examining the appearance of new subtypes of influenza-A virus type A shows that the antigenic variations occur for the most part as a result of mutations in this virus's neuraminidase and/or hemagglutinin genes.

In another aspect, the selected epitope 506 may not involve highly variable regions and may focus instead on areas having lower probability of mutations. Thus epitopes selected may avoid hot spots of antigenic variations and instead target other specific regions of an agent 400, such as, for example, the receptor-binding site on the surface of an agent 400. In another example, a selected epitope 506 may not be readily accessible to the immune response component, for example, the receptor-binding site may be buried deep in a ‘pocket’ of a large protein and may be surrounded by readily accessible sequences exhibiting higher level(s) of antigenic variations. In this example, one possibility may include providing small antibody fragments that penetrate the receptor binding site, thereby preventing an agent 400 from binding its target. In another example, a drug and/or chemical may be used to modify and/or enhance the accessibility of the receptor binding site. In yet another example, a chemical with a tag may be used to bind to the receptor and the tag then used for binding the immune response component.

In another aspect, the immune response component may be designed so as to circumvent the shape changes in an epitope 402 and provide sufficiently effective binding to an epitope 402 even following mutational change(s) therein. In this example, the antibody or other immune response component designed may include accommodations in its design deriving from the prediction of hot spots and/or the predicted mutational changes in an epitope 402. In one aspect, the size of the immune response component may be manipulated. An immune response component, for example, an antibody 404, may be designed to include the practicably minimal binding site required to bind an epitope 402. In another example, the immune response component may be designed for binding to the smallest effective determinant.

In one aspect, an effective treatment therapy for a disease and/or disorder may include one or more immune response components designed to anticipate and/or treat an antigenic drift and/or an antigenic shift predicted for multiple agents. The agents need not be related to each other; for example, the therapy might be designed for an individual suffering from multiple diseases.

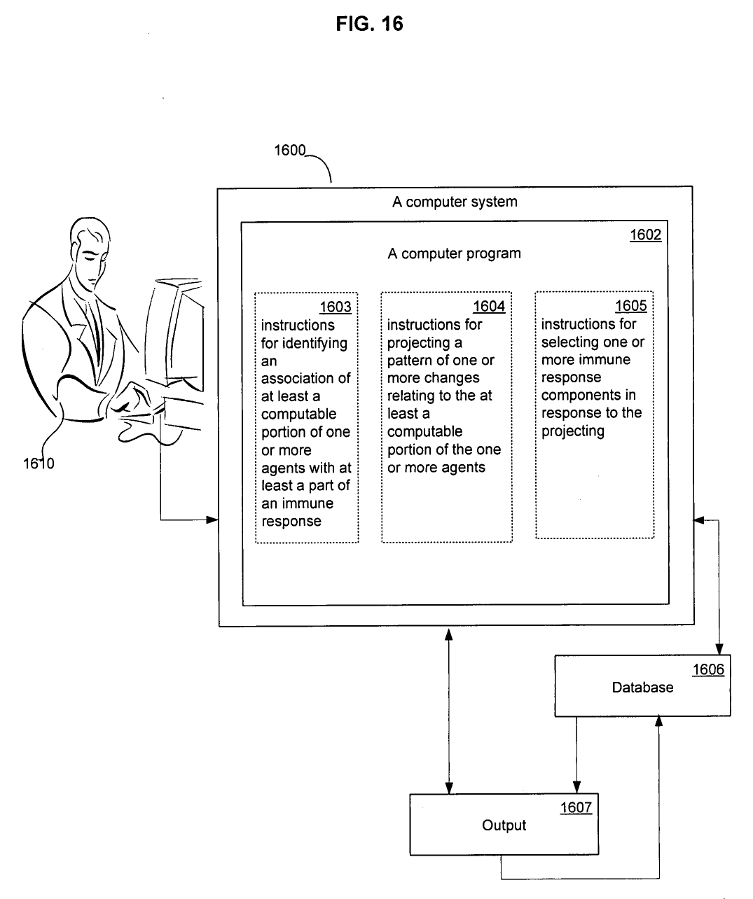

FIG. 16 depicts a partial view of a system that may serve as an illustrative environment of and/or for subject matter technologies. One or more users 1610 may use a computer system 1600 including a computer program 1602, for example, for identifying computable portions of an agent associated with a disease, disorder, or condition. The computer program 1602 may include one or more instructions, for example, instructions for identifying an association of at least a computable portion of one or more agents with at least a part of an immune response 1603, for example, identifying an association based on user defined parameters. The instructions may be such that, when they are loaded to a general purpose computer or microprocessor programmed to carry out the instructions, they create a new machine, because a general purpose computer in effect may become a special purpose computer once it is programmed to perform particular functions pursuant to instructions from program software. That is, the instructions of the software program may electrically change the general purpose computer by creating electrical paths within the device. These electrical paths, in some implementations, may create a special purpose machine having circuitry for carrying out the particular program. The computer program 1602 may include instructions 1604 that give rise to circuitry for projecting a pattern of one or more changes relating to the at least a computable portion of the one or more agents, for example, mutations, variations and/or alternate computable portions. The computer program 1602 may include instructions 1605 that give rise to circuitry for selecting one or more immune response components in response to the projecting, for example, including, but not limited to, a natural and/or a synthetic antibody. The computer program 1602 may accept input, for example, from medical personnel, a researcher, or wet lab personnel. A user interface may be coupled to provide access to the computer program 1602. In one implementation, the computer program 1602 may access a database 1606 for storing information and transmit an output 1607 to the computer system 1600. In one exemplary implementation, a feedback loop is set up between the computer program 1602 and the database 1606. The output 1607 may be fed back into the computer program 1602 and/or displayed on the computer system 1600. The system may be used as a research tool, as a tool for furthering treatment or the like. This feedback scheme may be useful in an iterative process such as described herein and elsewhere.

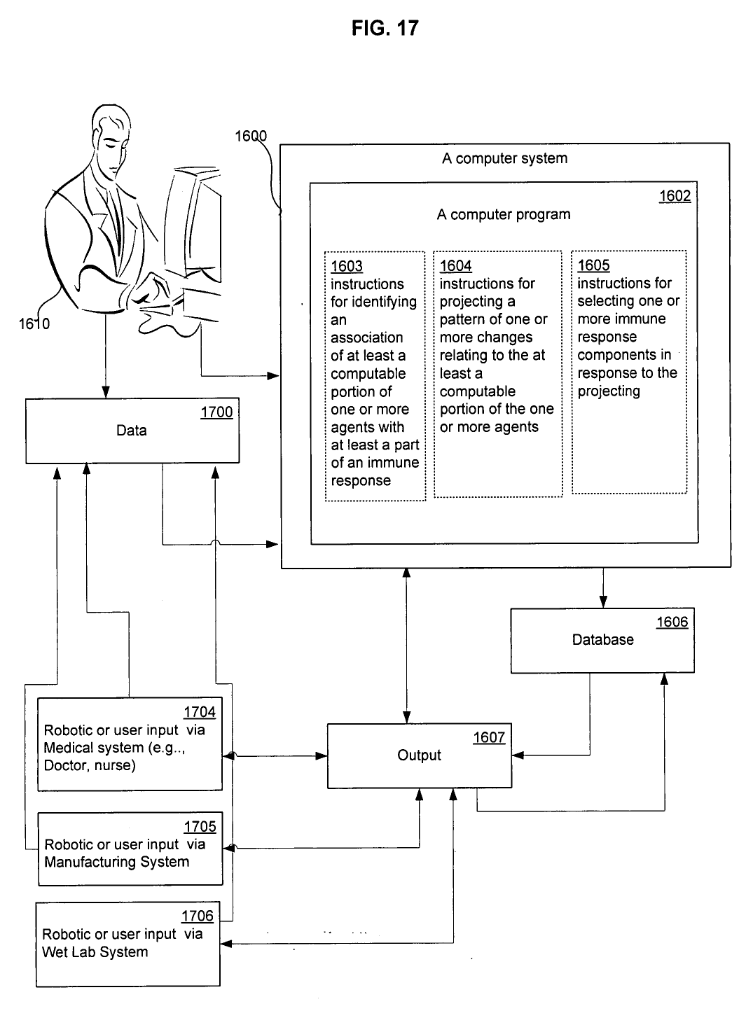

With reference to the figures, and with reference now to FIG. 17, depicted is a partial view of a system that may serve as an illustrative environment of and/or for subject matter technologies. The database 1606, data 1700, and/or the output 1607 may be accessed by various input mechanisms, for example, mechanisms including but not limited to, robotic and/or user input via a medical system 1704, robotic and/or user input via manufacturing system 1705, or robotic and/or user input via wet lab system 1706. Access to the data 1700 may be provided, for example, for further manipulation of the data.

In some aspects, a system may include circuitry for identifying an association of at least a computable portion of one or more agents with at least a part of an immune response, circuitry for projecting a pattern of one or more changes relating to the at least a computable portion of the one or more agents, and circuitry for selecting one or more immune response components in response to said circuitry for projecting. In some embodiments, the circuitry for selecting one or more immune response components may include circuitry for selecting at least a part of one or more of a T-lymphocyte, a killer T-lymphocyte, a helper T-lymphocyte, a cytotoxic T-lymphocyte, a T-8 lymphocyte, a Cluster of Differentiation (CD) molecule, a CD3 molecule, or a CD1 molecule. In some embodiments, the circuitry for selecting one or more immune response components may include circuitry for selecting one or more modulators of at least a part of at least one of a T-lymphocyte, a killer T-lymphocyte, a helper T-lymphocyte, a cytotoxic T-lymphocyte, a T-8 lymphocyte, a Cluster of Differentiation (CD) molecule, a CD3 molecule, or a CD 1 molecule. In some embodiments, the circuitry for selecting one or more immune response components may include circuitry for selecting at least a part of at least one B-lymphocyte, and/or circuitry for selecting one or more modulators of at least a part of at least one B-lymphocyte. In some embodiments, the circuitry for selecting one or more immune response components may include circuitry for selecting at least a part of one or more humanized antibody, and/or circuitry for selecting one or more modulators of at least a part of at least one humanized antibody. In some embodiments, the circuitry for identifying an association of at least a computable portion of one or more agents with at least a part of an immune response includes circuitry for identifying an association of at least a portion of at least one of a polyglycopeptide, a polysaccharide, a nucleic acid, or a toxin.

In some aspects, a system may include circuitry for accepting an input of one or more agents, and circuitry for identifying an association of at least one computable portion of one or more agents with at least a part of an immune response related to suppressing the one or more agents. In some aspects, a system may include circuitry for identifying an association of at least one antigen of one or more agents with at least a part of an immune response, circuitry for projecting a pattern of one or more changes relating to the at least one antigen of the one or more agents, and circuitry for selecting one or more immune response components responsive to said circuitry for projecting. In some aspects, a system may include identifying an association of at least one computable antigen of one or more agents with at least a part of an immune response, projecting a pattern of one or more changes relating to the at least one computable antigen of the one or more agents, and selecting one or more immune response components in response to the projecting. In some aspects, a system may include circuitry for associating at least one computable antigen of one or more agents with at least a part of an immune response, circuitry for projecting a pattern of one or more changes relating to the at least one computable antigen of the one or more agents, and circuitry for selecting one or more immune response components responsive to said circuitry for projecting. In some embodiments, a system includes circuitry for designating one or more computable epitopes of at least one agent, circuitry for predicting one or more changes in the one or more computable epitopes of the at least one agent, and circuitry for aiding the identification of one or more immune response components associated with the one or more computable epitopes of the at least one agent.

B. Operation(s) and/or Process(es)

Following are a series of flowcharts depicting implementations of processes. For ease of understanding, the logic flowcharts are organized such that the initial logic flowcharts present implementations via an overall “big picture” or “top-level” viewpoint and thereafter the following logic flowcharts present alternate implementations and/or expansions of the “big picture” logic flowcharts as either sub-steps or additional steps building on one or more earlier-presented logic flowcharts. Those having skill in the art will appreciate that the style of presentation utilized herein (e.g., beginning with a presentation of a logic flowchart(s) presenting an overall view of the system and thereafter providing additions to and/or further details in subsequent logic flowcharts) generally allows for a more rapid and reliable understanding of the various process implementations.

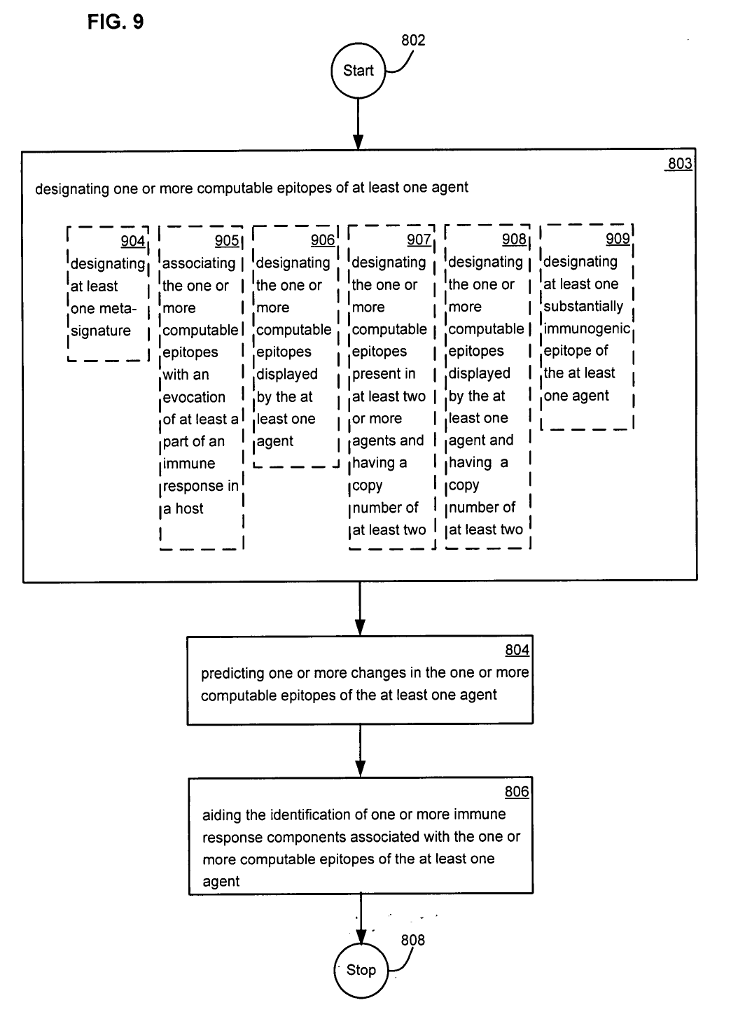

Several of the alternate process implementations are set forth herein by context. For example, as set forth herein in relation to FIG. 9, what is described as method step 803 is illustrated as a list of exemplary qualifications of designating one or more computable epitopes of at least one agent. Those skilled in the art will appreciate that when what is described as method-step 803 is read in context of what is described as method-step 904 it is apparent that the example set forth, in context, is actually illustrative of an alternate implementation of method step 803 of designating one or more computable epitopes of at least one agent such as, for example, designating at least one meta-signature. Contextual readings such as those just set forth in relation to method steps 904, and/or 803 are within the ambit of one having skill in the art in light of the teaching herein, and hence are not set forth verbatim elsewhere herein for sake of clarity.

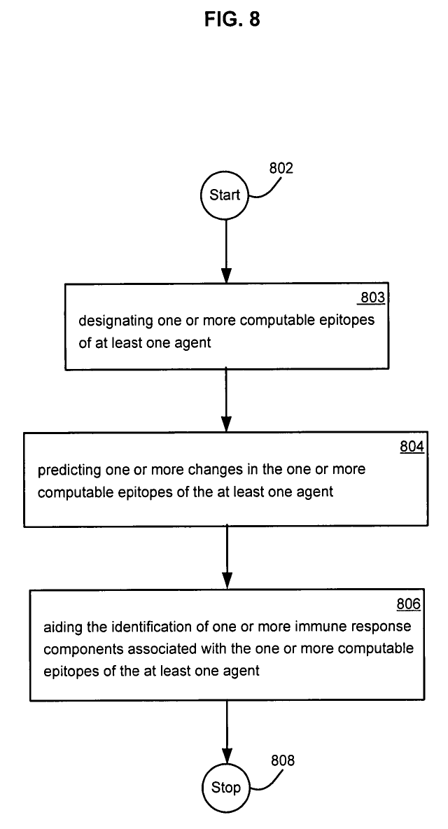

With reference now to FIG. 8, depicted is a high level logic flowchart of a process. Method step 802 shows the start of the process. Method step 803 depicts designating one or more computable epitopes of at least one agent. Method step 804 depicts predicting one or more changes in the one or more computable epitopes of the at least one agent. Method step 806 depicts aiding the identification of one or more immune response components associated with the one or more computable epitopes of the at least one agent. Method step 808 illustrates the end of the process. The one or more computable epitopes may include, for example, including but not limited to, at least a portion of an agent requiring management, at least a part of at least one of an amino acid, a nucleotide, a carbohydrate, a protein, a lipid, a capsid protein, a coat protein, a polysaccharide, a sugar, a lipopolysaccharide, a glycolipid, and/or a glycoprotein. It will be appreciated by those of skill in the art that the term “amino acid” may include, but is not limited to, complete and/or partial amino acids, amino acid residues, amino acid moieties, and/or components thereof. It will be appreciated by those of skill in the art that the term “nucleotide” may include, but is not limited to, complete and/or partial nucleotides, nucleotide residues, nucleotide moieties, nucleotide-mimetics, and/or components thereof. In one or more various implementations, one or more computer programs including instructions effecting one or more of the herein-described processes may be housed, for example, in a laptop computer system, on a server, a CD-ROM, DVD-ROM, or other electronically-readable media.

With reference now to FIG. 9, depicted is a high-level logic flowchart depicting alternate implementations of the high-level logic flowchart of FIG. 8. Illustrated is that in various alternate implementations, method step 803 may include at least one of method steps 904, 905, 906, 907, 908, and/or 909. Method step 904 depicts designating at least one meta-signature. Method step 905 depicts associating the one or more computable epitopes with an evocation of at least a part of an immune response in a host. Method step 906 depicts designating the one or more computable epitopes displayed by the at least one agent. Method step 907 depicts designating the one or more computable epitopes present in at least two or more agents and having a copy number of at least two. Method step 908 depicts designating the one or more computable epitopes displayed by the at least one agent and having a copy number of at least two. Method step 909 depicts designating at least one substantially immunogenic epitope of the at least one agent. It will also be appreciated by those skilled in the art that the method may include additional method steps, such as, for example, accepting input related to, for example, including, but not limited to, the agent, the one or more computable epitopes, size of the computable epitope, type of the computable epitope, type of disease, type of disorder, type of condition requiring management, and/or sensitivity (e.g., hypersensitivity and/or allergic reactions) of the group requiring management.

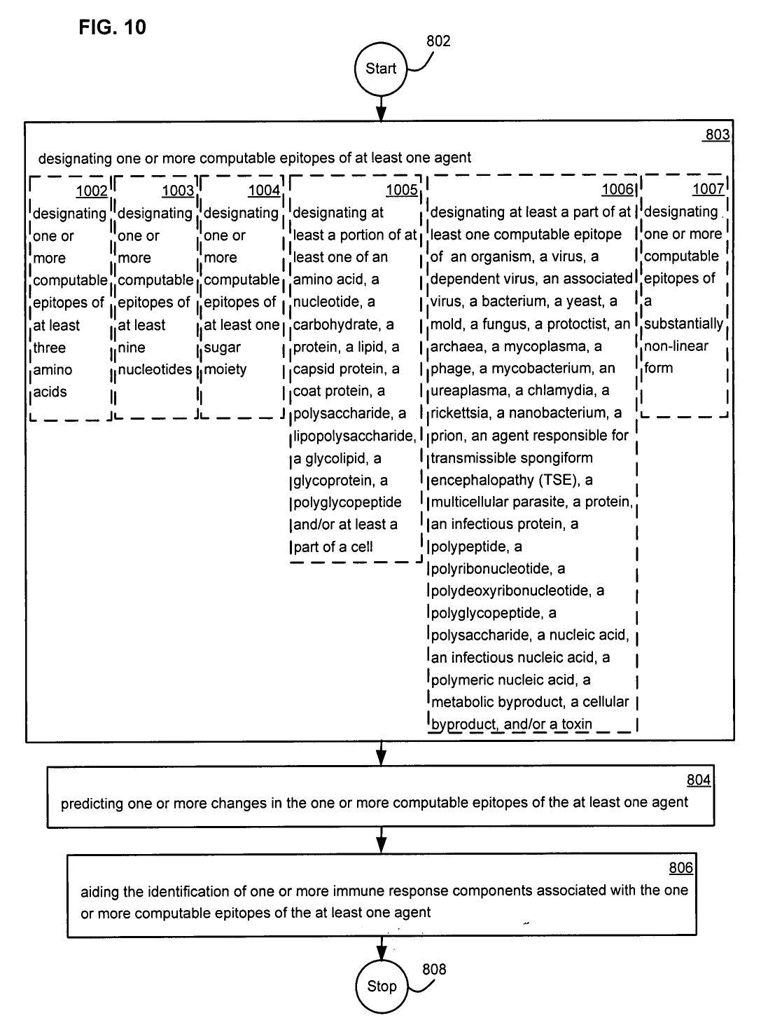

With reference now to FIG. 10, depicted is a high-level logic flowchart depicting alternate implementations of the high-level logic flowchart of FIG. 8. Illustrated is that in various alternate implementations, method step 803 may include at least one of method steps 1002, 1003, 1004, 1005, 1006 and/or 1007. Method step 1002 depicts designating one or more computable epitopes of at least three amino acids. Method step 1003 depicts designating one or more computable epitopes of at least nine nucleotides. Method step 1004 depicts designating one or more computable epitopes of at least one sugar moiety. Method step 1005 depicts designating at least a portion of at least one of an amino acid, a nucleotide, a carbohydrate, a protein, a lipid, a capsid protein, a coat protein, a polysaccharide, a lipopolysaccharide, a glycolipid, a glycoprotein, a polyglycopeptide, and/or at least a part of a cell. Method step 1005 depicts designating at least a part of at least one computable epitope of an organism, a virus, a dependent virus, an associated virus, a bacterium, a yeast, a mold, a fungus, a protoctist, an archaea, a mycoplasma, a phage, a mycobacterium, an ureaplasma, a chlamydia, a rickettsia, a nanobacterium, a prion, an agent responsible for transmissible spongiform encephalopathy (TSE), a multicellular parasite, a protein, an infectious protein, a polypeptide, a polyribonucleotide, a polydeoxyribonucleotide, a polyglycopeptide, a polysaccharide, a nucleic acid, an infectious nucleic acid (e.g., an infectious nucleic acid polymer), a polymeric nucleic acid, a metabolic byproduct, a cellular byproduct, and/or a toxin. Method step 1006 depicts designating one or more computable epitopes of a substantially non-linear form.

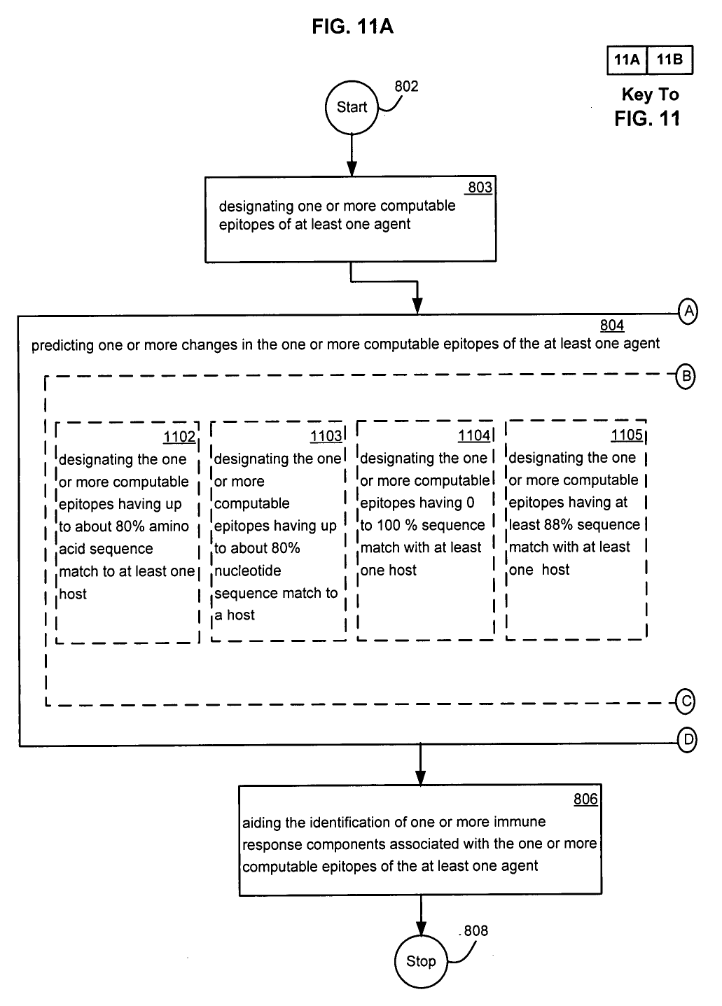

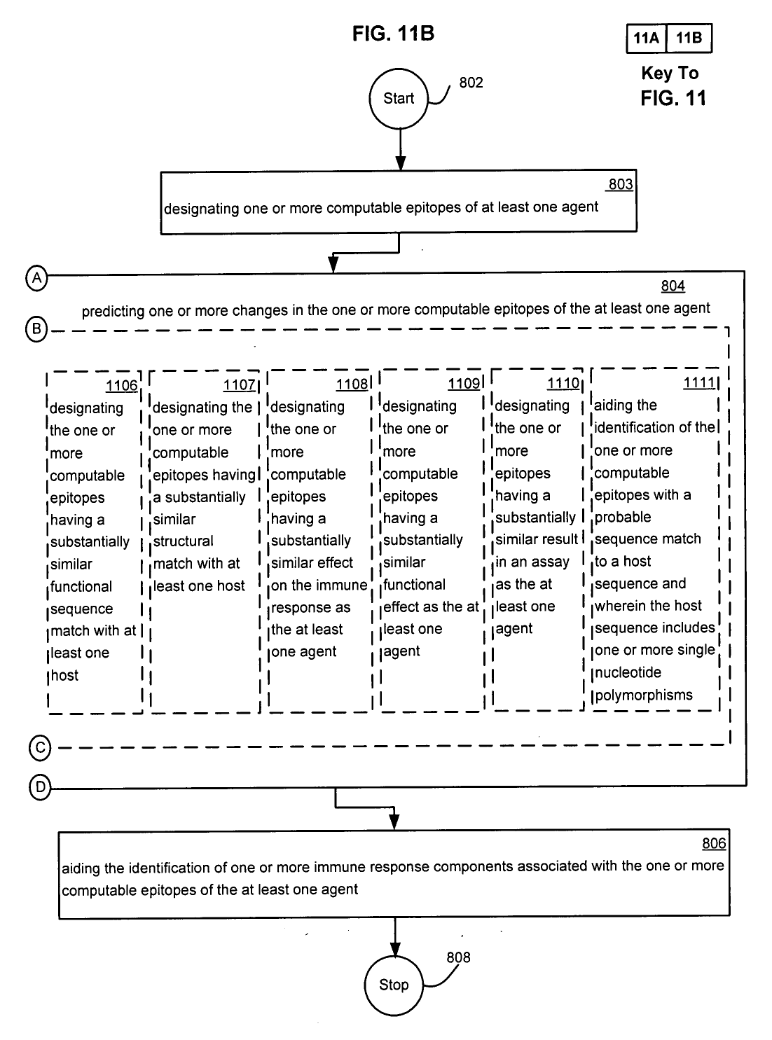

With reference now to FIG. 11, depicted is a high-level logic flowchart depicting alternate implementations of the high-level logic flowchart of FIG. 8. Illustrated is that in various alternate implementations, method step 804 may include at least one of method steps 1102, 1103, 1104, 1105, 1106, 1107, 1108, 1109, 1110, and/or 1111. Method step 1102 depicts designating the one or more computable epitopes having up to about 80% amino acid sequence match to at least one host. Method step 1103 depicts designating the one or more computable epitopes having up to about 80% nucleotide sequence match to a host. Method step 1104 depicts designating the one or more computable epitopes having between 0 to 100% sequence match with at least one host (e.g., a 0% practicable sequence match is sometimes useful, for example, in implementations including, but not limited to, when the sequence desired is one that elicits a practicably relatively lower auto-immune response in the host and/or when the sequence desired is one that has a relatively lower crossover with sequences of another agent; a 100% practicable sequence match is sometimes useful, for example, in implementations including, but not limited to, when the sequence desired is one that elicits a practicably higher immune response in the host against the agent, and/or when the sequence desired is one that has a practicably relatively higher crossover sequence match with the host (e.g., an irretrievably infected host), for example, when eradication of the host needs to be accomplished in an environmentally-friendly manner). Method step 1105 depicts designating the one or more computable epitopes having at least 88% sequence match with at least one host. Method step 1106 depicts designating the one or more computable epitopes having a substantially similar functional sequence match with at least one host. Method step 1107 depicts designating the one or more computable epitopes having a substantially similar structural match with at least one host. Method step 1108 depicts designating the one or more computable epitopes having a substantially similar effect on the immune response as the at least one agent. Method step 1109 depicts designating the one or more computable epitopes having a substantially similar functional effect as the at least one agent. Method step 1110 depicts designating the one or more epitopes having a substantially similar result in an assay as the at least one agent. Method step 1111 depicts aiding the identification of the one or more computable epitopes with a probable sequence match to a host sequence and wherein the host sequence includes one or more single nucleotide polymorphisms.

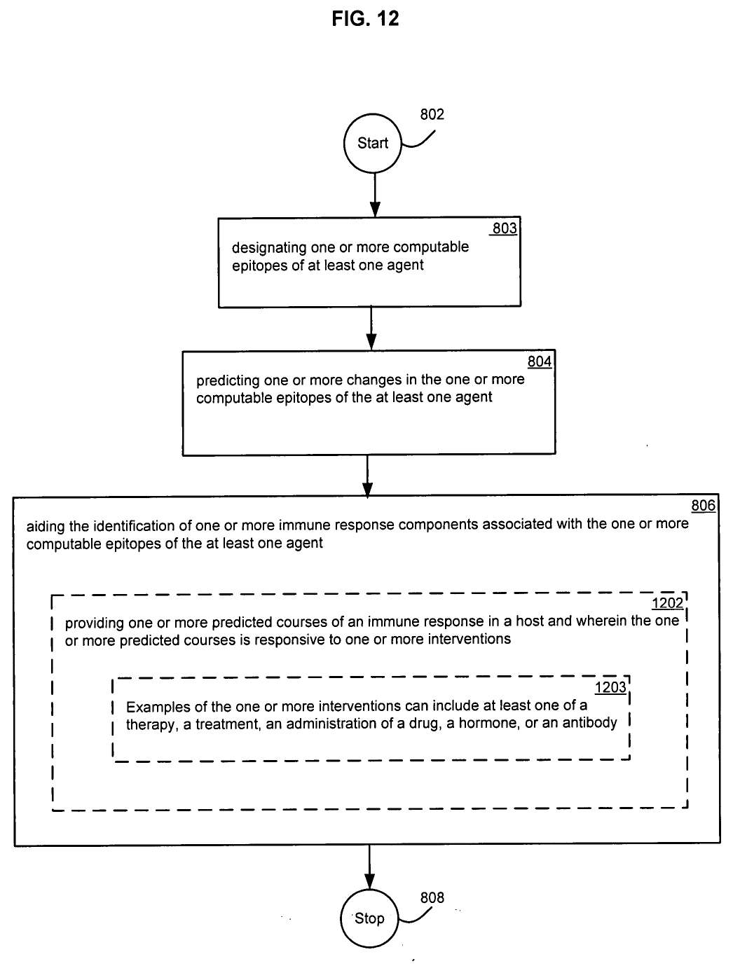

With reference now to FIG. 12, depicted is a high-level logic flowchart depicting alternate implementations of the high-level logic flowchart of FIG. 8. Illustrated is that in various alternate implementations, method step 806 may include method step 1202. Method step 1202 depicts providing one or more predicted courses of an immune response in a host and wherein the one or more predicted courses are responsive to one or more interventions. Depicted is that in one alternate implementation method step 1202 may include example-block 1203 which sets forth examples of the one or more interventions. Example-block 1203 depicts that examples of the one or more interventions may include at least one of a therapy, a treatment, an administration of a drug, a hormone, and/or an antibody.

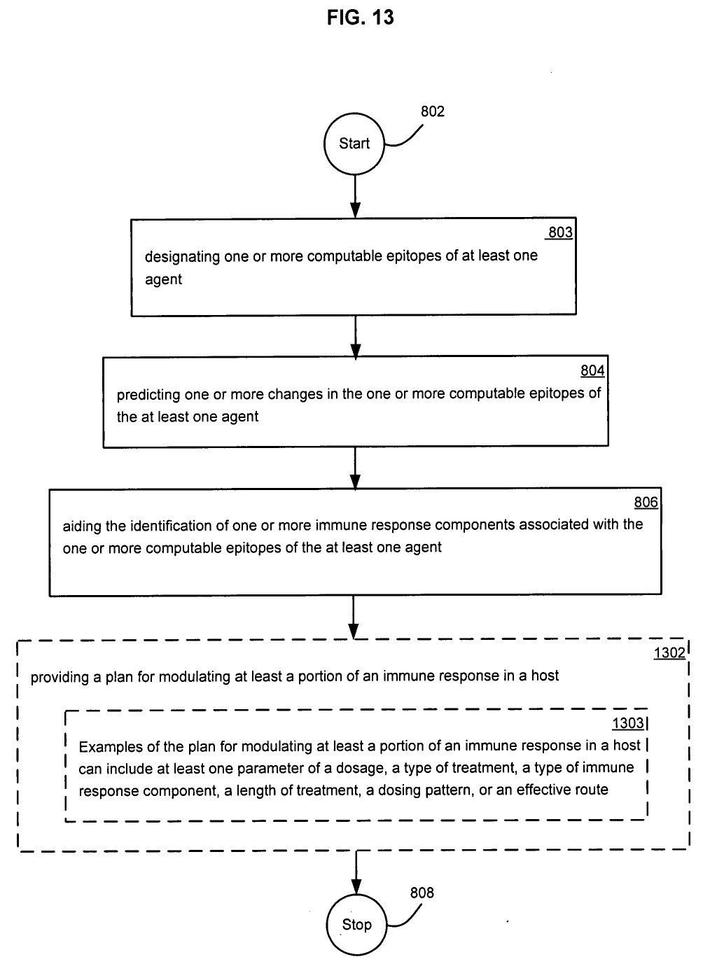

With reference now to FIG. 13, depicted is a high-level logic flowchart depicting alternate implementations of the high-level logic flowchart of FIG. 8. Illustrated is that in various alternate implementations, the process may include method step 1302. Method step 1302 depicts providing a plan for modulating at least a portion of an immune response in a host. Depicted is that in one alternate implementation method step 1302 may include example-block 1303 which sets forth examples of the plan. Example-block 1303 depicts that examples of the plan for modulating may include at least one parameter of a dosage, a type of treatment, a type of immune response component, a length of treatment, a dosing pattern, and/or an effective route.

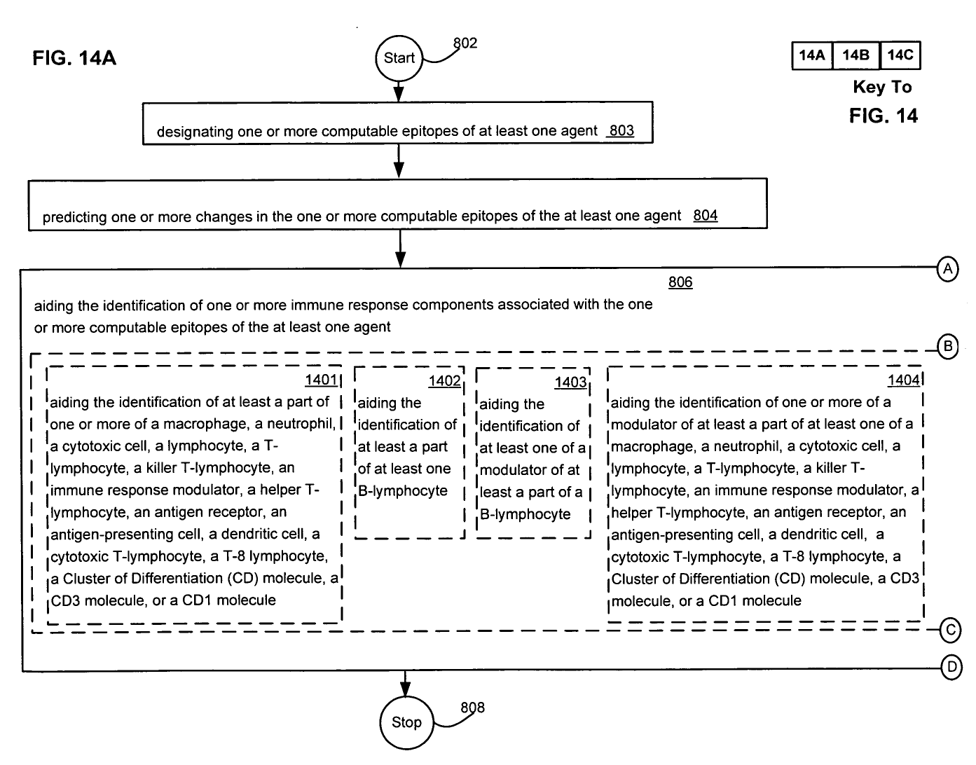

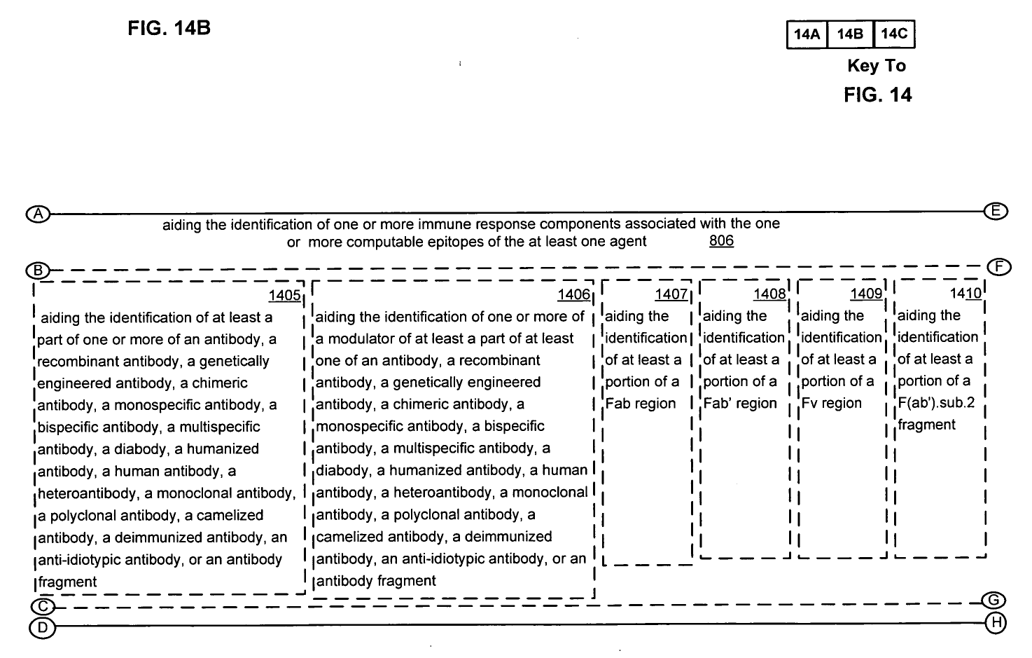

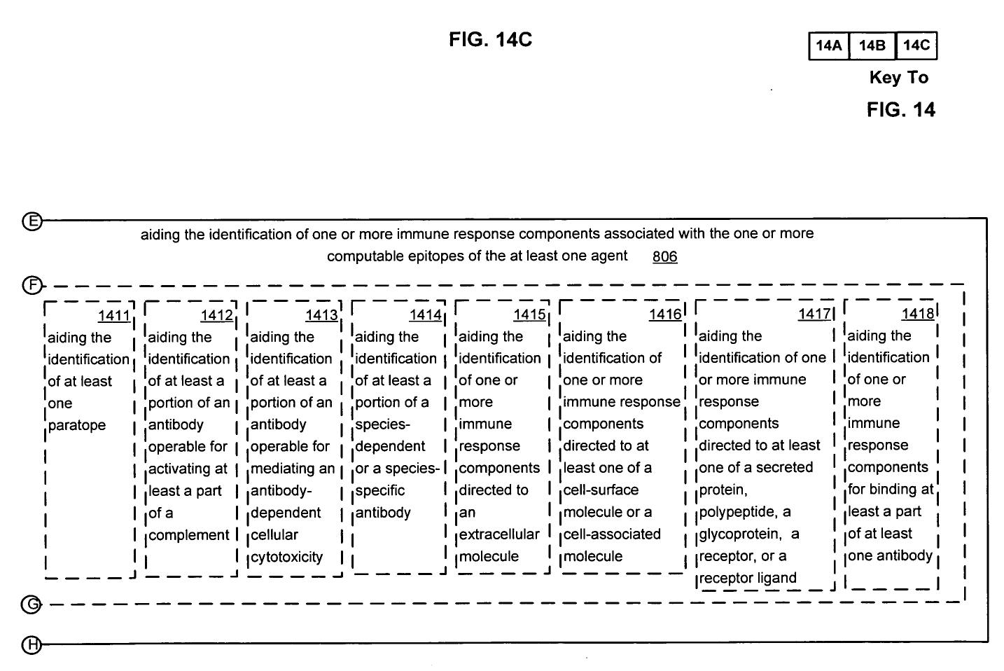

With reference now to FIG. 14, depicted is a high-level logic flowchart depicting alternate implementations of the high-level logic flowchart of FIG. 8. Illustrated is that in various alternate implementations, method step 806 may include at least one of method steps 1401, 1402, 1403, 1404, 1405, 1406, 1407, 1408, 1409, 1410, 1411, 1412, 1413, 1414, 1415, 1416, 1417, and/or 1418. Method step 1401 depicts aiding the identification of at least a part of one or more of a macrophage, a neutrophil, a cytotoxic cell, a lymphocyte, a T-lymphocyte, a killer T-lymphocyte, an immune response modulator, a helper T-lymphocyte, an antigen receptor, an antigen-presenting cell, a dendritic cell, a cytotoxic T-lymphocyte, a T-8 lymphocyte, a Cluster of Differentiation (CD) molecule, a CD3 molecule, and/or a CD1 molecule. Method step 1402 depicts aiding the identification of at least a part of at least one B-lymphocyte. Method step 1403 depicts aiding the identification of at least one modulator of at least a part of a B-lymphocyte. Method step 1404 depicts aiding the identification of one or more of a modulator of at least a part of at least one of a macrophage, a neutrophil, a cytotoxic cell, a lymphocyte, a T-lymphocyte, a killer T-lymphocyte, an immune response modulator, a helper T-lymphocyte, an antigen receptor, an antigen-presenting cell, a dendritic cell, a cytotoxic T-lymphocyte, a T-8 lymphocyte, a Cluster of Differentiation (CD) molecule, a CD3 molecule, and/or a CD1 molecule. Method step 1405 depicts aiding the identification of at least a part of one or more of an antibody, a recombinant antibody, a genetically engineered antibody, a chimeric antibody, a monospecific antibody, a bispecific antibody, a multispecific antibody, a diabody, a humanized antibody, a human antibody, a heteroantibody, a monoclonal antibody, a polyclonal antibody, a camelized antibody, a deimmunized antibody, an anti-idiotypic antibody, or an antibody fragment. Method step 1406 depicts aiding the identification of one or more of a modulator of at least a part of at least one of an antibody, a recombinant antibody, a genetically engineered antibody, a chimeric antibody, a monospecific antibody, a bispecific antibody, a multispecific antibody, a diabody, a humanized antibody, a human antibody, a heteroantibody, a monoclonal antibody, a polyclonal antibody, a camelized antibody, a deimmunized antibody, an anti-idiotypic antibody, and/or an antibody fragment. Method step 1407 depicts aiding the identification of at least a portion of a Fab region. Method step 1408 depicts instructions for aiding the identification of at least a portion of a Fab′ region. Method step 1409 depicts aiding the identification of at least a portion of a Fv region. Method step 1410 depicts aiding the identification of at least a portion of a F(ab′).sub.2 fragment. Method step 1411 depicts aiding the identification of at least one paratope. Method step 1412 depicts aiding the identification of at least a portion of an antibody operable for activating at least a part of a complement. Method step 1413 depicts aiding the identification of at least a portion of an antibody operable for mediating an antibody-dependent cellular cytotoxicity. Method step 1414 depicts aiding the identification of at least a portion of a species-dependent and/or species-specific antibody. Method step 1415 depicts aiding the identification of one or more immune response components directed to an extracellular molecule. Method step 1416 depicts aiding the identification of one or more immune response components directed to at least one of a cell-surface molecule and/or a cell-associated molecule. Method step 1417 depicts aiding the identification of one or more immune response components directed to at least one of a secreted protein, a polypeptide, a glycoprotein, a receptor, and/or a receptor ligand. Method step 1418 depicts aiding the identification of one or more immune response components for binding at least a part of at least one antibody.

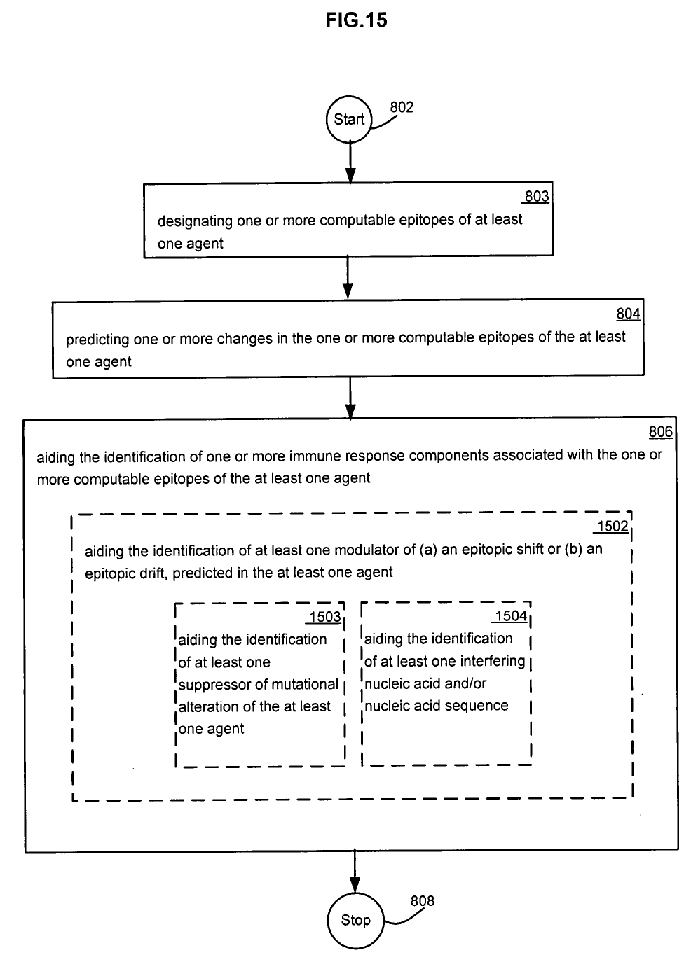

With reference now to FIG. 15, depicted is a high-level logic flowchart depicting alternate implementations of the high-level logic flowchart of FIG. 8. Illustrated is that in various alternate implementations, method step 806 may include method step 1502. Method step 1502 depicts aiding the identification of at least one modulator of (a) an epitopic shift or (b) an epitopic drift that is predicted in the at least one agent (for example, the epitopic shift and/or drift may be computable, compositional and/or structural). Illustrated is that in various alternate implementations, method step 1502 may include at least one of method steps 1503, and/or 1504. Method step 1503 depicts aiding the identification of at least one suppressor of mutational alteration of the at least one agent (e.g., an escape mutation for down-regulating and/or up-regulating a gene and/or a related gene activity). Method step 1504 depicts aiding the identification of at least one interfering nucleic acid and/or nucleic acid sequence (e.g., one or more of a deoxynucleotide, a chemically synthesized nucleotide, a nucleotide analog, a nucleotide not naturally occurring, or a nucleotide not found in natural RNA or DNA of an untreated agent) and/or a (e.g., polymerized) set of such nucleic acids.

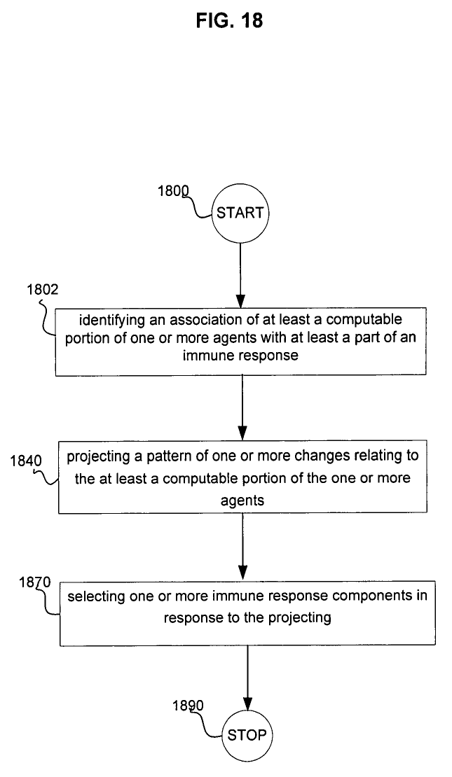

With reference now to FIG. 18, depicted is a high-level logic flowchart of a process. Method step 1800 shows the start of the process. Method step 1802 depicts identifying an association of at least a computable portion of one or more agents with at least a part of an immune response. The computable portion may, for example, may include, but is not limited to, at least a portion of an agent requiring management. The computable portion may also include, at least 3 amino acids, a sequence of at least nine nucleotides, at least a part of at least one of an amino acid, a nucleotide, a carbohydrate, a protein, a lipid, a capsid protein, a coat protein, a polysaccharide, a lipopolysaccharide, a glycolipid, a glycoprotein, and/or at least a part of a cell or a biological entity (e.g., a virus particle). It will be appreciated by those of skill in the art that the term “amino acid” may include, but is not limited to, complete and/or partial amino acids, amino acid residues, amino acid moieties, and/or components thereof. It will be appreciated by those of skill in the art that the term “nucleotide” may include, but is not limited to, complete and/or partial nucleotides (including artificial and/or synthetic nucleotides and/or nucleotide-mimetics), nucleotide residues, nucleotide moieties, and/or components thereof. Method step 1840 depicts projecting a pattern of one or more changes relating to the at least a computable portion of the one or more agents. Method step 1870 depicts selecting one or more immune response components in response to the projecting. Method step 1890 shows the culmination of the process. It will also be appreciated by those skilled in the art that method steps 1800, 1802, 1840, 1870, and/or 1890 may include accepting input related to, for example, the agent, and/or the one or more computable epitopes. Examples of criteria related to the agent and/or the computable epitopes may include, but is not limited to, size of the computable epitope, type of the computable epitope, nature of the disease, nature of disorder, nature of condition requiring management, and/or a sensitivity of the group requiring management. Portions of the disclosure herein (e.g., flowcharts and/or supporting descriptions and/or claims) refer to “computable portion(s).” Such portions can be modified to refer to and teach computable epitope(s), epitope(s), and/or computable antigen(s), as appropriate, especially in light of the teachings of the as-filed claims. Such modifications of the portions are within the ambit of those skilled in the art, and hence are not expressly set forth herein for sake of clarity. Furthermore, those skilled in the art will appreciate that, in general, computable portions, computable epitopes, antigens, and/or computable antigens may be indicative of a part, a section, and/or a whole and may also be illustrative of a predicted, original, or mutable sequence (e.g., a sequence including an amino acid, a nucleotide and/or a sugar) unless context dictates otherwise.

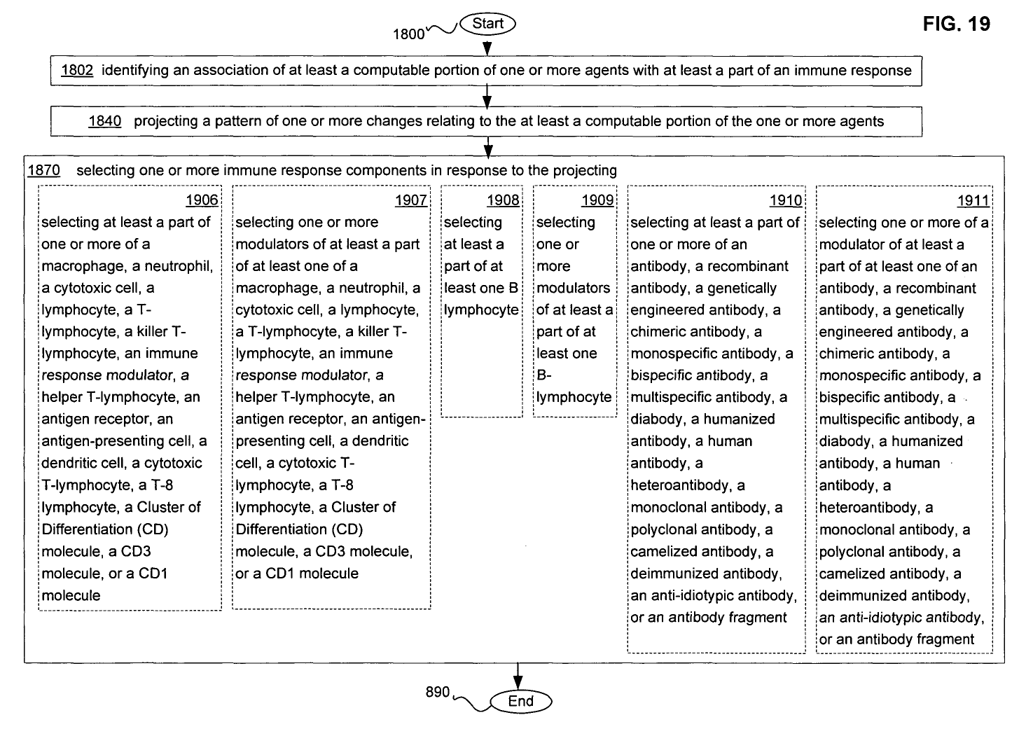

With reference now to FIG. 19, depicted is a high-level logic flowchart depicting alternate implementations of the high-level logic flowchart of FIG. 18. Illustrated is that in various alternate implementations, method step 1870 may include at least one of substeps 1906, 1907, 1908, 1909, 1910, and/or 1911. Method step 1906 depicts selecting at least a part of one or more of a macrophage, a neutrophil, a cytotoxic cell, a lymphocyte, a T-lymphocyte, a killer T-lymphocyte, an immune response modulator, a helper T-lymphocyte, an antigen receptor, an antigen-presenting cell, a dendritic cell, a cytotoxic T-lymphocyte, a T-8 lymphocyte, a Cluster of Differentiation (CD) molecule, a CD3 molecule, and/or a CD1 molecule. Method step 1907 depicts selecting one or more modulators of at least a part of at least one of a macrophage, a neutrophil, a cytotoxic cell, a lymphocyte, a T-lymphocyte, a killer T-lymphocyte, an immune response modulator, a helper T-lymphocyte, an antigen receptor, an antigen-presenting cell, a dendritic cell, a cytotoxic T-lymphocyte, a T-8 lymphocyte, a Cluster of Differentiation (CD) molecule, a CD3 molecule, and/or a CD1 molecule. Method step 1908 depicts selecting at least a part of at least one B-lymphocyte. Method step 1909 depicts selecting one or more of modulators of at least a part of at least one B-lymphocyte. Method step 1910 depicts selecting at least a part of one or more of an antibody, a recombinant antibody, a genetically engineered antibody, a chimeric antibody, a monospecific antibody, a bispecific antibody, a multispecific antibody, a diabody, a humanized antibody, a human antibody, a heteroantibody, a monoclonal antibody, a polyclonal antibody, a camelized antibody, a deimmunized antibody, an anti-idiotypic antibody, and/or an antibody fragment. Method step 1911 depicts selecting one or more of a modulator of at least a part of at least one of an antibody, a recombinant antibody, a genetically engineered antibody, a chimeric antibody, a monospecific antibody, a bispecific antibody, a multispecific antibody, a diabody, a humanized antibody, a human antibody, a heteroantibody, a monoclonal antibody, a polyclonal antibody, a camelized antibody, a deimmunized antibody, an anti-idiotypic antibody, and/or an antibody fragment.

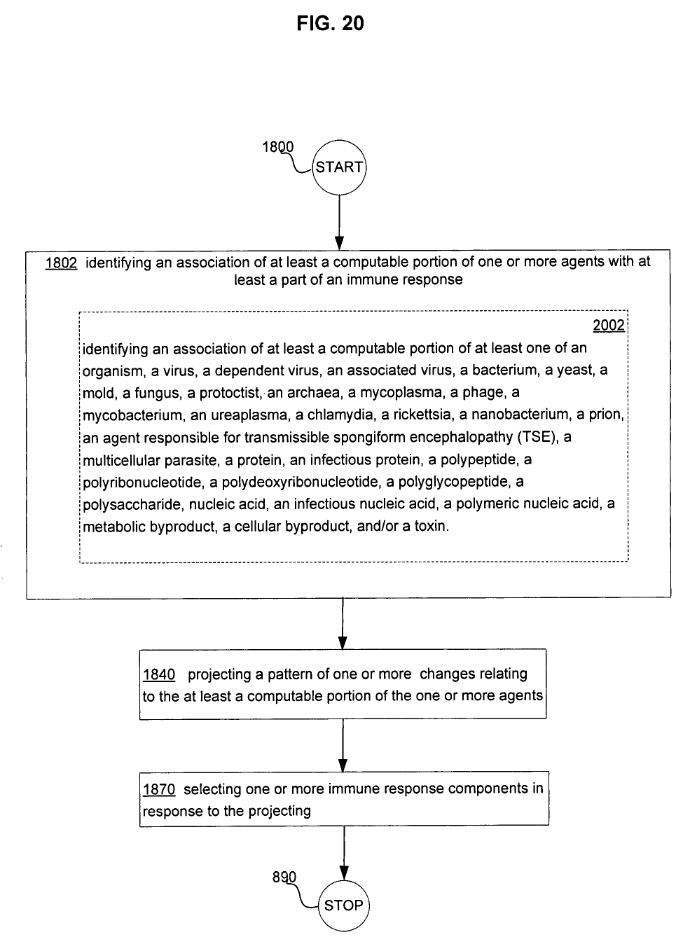

With reference now to FIG. 20, depicted is a high-level logic flowchart depicting alternate implementations of the high-level logic flowchart of FIG. 18. In some aspects, method step 1802 may include method step 2002, identifying an association of at least a computable portion of at least one of an organism, a virus, a dependent virus, an associated virus, a bacterium, a yeast, a mold, a fungus, a protoctist, an archaea, a mycoplasma, a phage, a mycobacterium, an ureaplasma, a chlamydia, a rickettsia, a nanobacterium, a prion, an agent responsible for transmissible spongiform encephalopathy (TSE), a multicellular parasite, a protein, an infectious protein, a polypeptide, a polyribonucleotide, a polydeoxyribonucleotide, a polyglycopeptide, a polysaccharide, nucleic acid, an infectious nucleic acid, a polymeric nucleic acid, a metabolic byproduct, a cellular byproduct, and/or a toxin.

In some aspects, a method may include identifying an association of at least one computable epitope of one or more agents with at least a part of an immune response, projecting a pattern of one or more changes relating to the at least one computable epitope of the one or more agents, and selecting one or more immune response components in response to the projecting. In some aspects, selecting one or more immune response components in response to the projecting may include selecting at least a part of one or more of a T-lymphocyte, a killer T-lymphocyte, a helper T-lymphocyte, a cytotoxic T-lymphocyte, a T-8 lymphocyte, a Cluster of Differentiation (CD) molecule, a CD3 molecule, or a CD1 molecule. In some aspects, selecting one or more immune response components in response to the projecting may include selecting one or more modulators of at least a part of at least one of a T-lymphocyte, a killer T-lymphocyte, a helper T-lymphocyte, a cytotoxic T-lymphocyte, a T-8 lymphocyte, a Cluster of Differentiation (CD) molecule, a CD3 molecule, or a CD1 molecule. In some aspects, selecting one or more immune response components in response to the projecting may include selecting at least a part of at least one B-lymphocyte, and/or selecting one or more modulators of at least a part of at least one B-lymphocyte. In some aspects, selecting one or more immune response components in response to the projecting may include selecting at least a part of one or more humanized antibody, and/or one or more modulators of at least a part of at least one humanized antibody. In some aspects, identifying an association of at least one computable epitope of one or more agents with at least a part of an immune response may include identifying an association of at least one computable epitope of at least one of a polyglycopeptide, a polysaccharide, a nucleic acid, or a toxin.

In some aspects, a method may include accepting an input of one or more agents; and identifying an association of at least one computable portion of one or more agents with at least a part of an immune response related to suppressing the one or more agents. In some aspects, a method may include identifying an association of at least one antigen of one or more agents with at least a part of an immune response, projecting a pattern of one or more changes relating to at least one antigen of the one or more agents, and selecting one or more immune response components in response to the projecting. In some aspects, a method may include identifying an association of at least one epitope of one or more agents with at least a part of an immune response, projecting a pattern of one or more changes relating to the at least one epitope of the one or more agents, and selecting one or more immune response components in response to the projecting. In some aspects, a method may include identifying an association of at least one computable antigen of one or more agents with at least a part of an immune response, projecting a pattern of one or more changes relating to the at least one computable antigen of the one or more agents, and selecting one or more immune response components in response to the projecting.

C. Variation(s), and/or Implementation(s)

Those having skill in the art will recognize that the present application teaches modifications of the devices, structures, and/or processes within the spirit of the teaching herein. For example, in one aspect, the immune response components may be formulated to cross the blood-brain barrier which is known to exclude mostly hydrophilic compounds, as well as to discriminate against transport of high molecular weight ones. For example, an antibody fragment may be encased in a lipid vesicle. In another example, the antibody or a portion of the antibody may be tagged onto a carrier protein or molecule. In another example, an antibody or other immune response component may be split into one or more complementary fragments, each fragment encased by a lipid vesicle, and each fragment functional only on binding its complementary fragment. Once the blood-brain barrier has been crossed, the lipid vesicle may be dissolved to release the antibody fragments which reunite with their complementary counterparts and may form a fully functional antibody or other immune response component. Other modifications of the subject matter herein will be appreciated by one of skill in the art in light of the teachings herein.

Those having skill in the art will recognize that the present application teaches modifications of the devices, structures, and/or processes within the spirit of the teaching herein. For example, in one aspect, the immune response components may be made in large format. The method lends itself to both small format or personalized care applications and large scale applications or large format applications. Other modifications of the subject matter herein will be appreciated by one of skill in the art in light of the teachings herein.

Those having skill in the art will recognize that the present application teaches modifications of the devices, structures, and/or processes within the spirit of the teaching herein. For example, in one aspect, the method may be used to designate immune response components for any disease or disorder. The application of this method is not limited to diseases where antigenic shift or drift keeps the immune system “guessing” causing it to be effectively slow-to-respond or to be incapable of effective response. Although, influenza-A or HIV-1 are likely viral-disease-agent candidates, for application of this method, treatment of other diseases, disorders and/or conditions will likely benefit from this methodology. Other modifications of the subject matter herein will be appreciated by one of skill in the art in light of the teachings herein.

Those having skill in the art will recognize that the present application teaches modifications of the devices, structures, and/or processes within the spirit of the teaching herein. For example, in one aspect, real-time evaluation may be provided of the antigenic changes by including a portable PCR-enabled machine which samples the environment for (sub-) strains of pathogens locally present. The information may be sent remotely to another location or to a portable material-administering device, for example, a drip-patch device with a remote sensor, utilized by a potentially-affected host, resulting in the activation of the necessary immune response components and thereby providing adequate protection to the potential host. As the evaluation possibly changes in time, the portable material-administering device may be triggered to change the dosage or type of immune response component delivered. Such a portable material-administering device operably coupled to a portable PCR-enabled machine or a fully functional system has a wide variety of applications, for example, including, but not limited to, when medical personnel visit an area in which a disease is endemic, and/or when military personnel enter territory in which unknown pathogens may be present. Other modifications of the subject matter herein will be appreciated by one of skill in the art in light of the teachings herein.

Those having skill in the art will recognize that the present application teaches modifications of the devices, structures, and/or processes within the spirit of the teaching herein. For example, in one aspect, a potential host may use a portable material-administering device infused with the immune response components preprogrammed to provide the potential host with immune response-mediated protection over an interval of time, and/or to anticipate pattern changes in epitopes of an agent. For example, in one aspect, an individual may use an administering device including the immune response components preprogrammed to provide the user the necessary immune response-mediated protection over an interval period of time, and/or to anticipate pattern changes in the epitopes of an agent. Other modifications of the subject matter herein will be appreciated by one of skill in the art in light of the teachings herein.

Those having skill in the art will recognize that the present application teaches modifications of the devices, structures, and/or processes within the spirit of the teaching herein. For example, in one aspect, RNA blockers, and/or RNAi technology may be used to down-regulate genes or interfere productively with their expression, or to otherwise usefully modulate components of the immune system in conjunction with the method. Other modifications of the subject matter herein will be appreciated by one of skill in the art in light of the teachings herein.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

Those having skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency vs. operational convenience tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some particular combination of hardware, software, and/or firmware. Hence, there are several possible vehicles by which the processes and/or devices and/or other technologies described herein may be effected, none of which is inherently and universally superior to any other, in that any vehicle to be utilized is a choice dependent upon the context in which the vehicle will be deployed and the specific concerns (e.g., speed, flexibility, or predictability) of the implementer, any of which may vary substantially.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), other integrated formats, or other extensively-integrated formats. However, those skilled in the art will recognize that some aspects of the embodiments disclosed herein, in whole or in part, can be equivalently implemented in standard integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter subject matter described herein applies equally regardless of the particular type of signal-bearing media used to actually carry out the distribution. Examples of a signal-bearing media include, but are not limited to, the following: recordable type media such as floppy disks, hard disk drives, DVD/CD-ROMs, digital tape, computer memory devices of various types; and data transmission type-media; such as digital and analog communication links using TDM or IP-based communication links (e.g., packetized data links).

In a general sense, those skilled in the art will recognize that the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application-specific integrated circuit, electrical circuitry forming a general-purpose computing device configured by a computer program (e.g., a general-purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment).

Those skilled in the art will recognize that it is common within the art to describe devices and/or processes in the fashion set forth herein, and thereafter use standard engineering practices to integrate such described devices and/or processes into data-processing systems. That is, at least a portion of the devices and/or processes described herein can be integrated into a data processing system via a reasonable amount of experimentation. Those having skill in the art will recognize that a typical data-processing system generally includes one or more of a system unit housing, a video display device, a display device, a memory such as volatile and/or non-volatile memory, processors such as microprocessors and digital signal processors, computational entities such as operating systems, drivers, user interfaces (e.g., graphical), and applications programs, one or more interaction devices, such as a touch pad or screen, and/or control systems including feedback loops and control motors (e.g., feedback for sensing position and/or velocity; control motors for moving and/or adjusting components such as valves and/or quantities). A typical data processing system may be implemented utilizing any suitable commercially available components, such as those typically found in digital data computing/communication and/or network computing/communication systems.

All of the referenced U.S. patents, U.S. patent application publications, U.S. patent applications, foreign patents, foreign patent applications, and/or non-patent publications referred to in this specification and/or listed in any Application Data Sheet, are incorporated herein by reference, in their entireties.

The herein described aspects depict different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components.

While particular aspects of the present subject matter described herein have been shown and described, it will be apparent to those skilled in the art that, based upon the teachings herein, changes and modifications may be made without departing from this subject matter described herein and its broader aspects and, therefore, the appended claims are to encompass within their scope all such changes and modifications as are within the true spirit and scope of this subject matter described herein. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.).

1. A method, comprising:
designating one or more computable epitopes of at least one agent;
predicting one or more changes in the one or more computable epitopes of the at least one agent; and
aiding the identification of one or more immune response components associated with the one or more computable epitopes of the at least one agent.

2. The method of claim 1, wherein the designating one or more computable epitopes of at least one agent further comprises:
designating one or more computable epitopes of at least nine nucleotides.

3. The method of claim 1, wherein the designating one or more computable epitopes of at least one agent further comprises:
designating at least a portion of at least one of a nucleotide, a carbohydrate, a lipid, a polysaccharide, a lipopolysaccharide, a glycolipid, a glycoprotein, and/or a polyglycopeptide.

4. The method of claim 1, wherein the designating one or more computable epitopes of at least one agent further comprises:
designating one or more computable epitopes of a substantially non-linear form.

5. (canceled)

6. (canceled)

7. (canceled)

8. (canceled)

9. The method of claim 1, wherein the predicting one or more changes in the one or more computable epitopes of the at least one agent further comprises:
designating the one or more computable epitopes having a substantially similar functional sequence match with at least one host.

10. The method of claim 1, wherein the predicting one or more changes in the one or more computable epitopes of the at least one agent further comprises:
designating the one or more computable epitopes having a substantially similar structural match with at least one host.

11. The method of claim 1, wherein the aiding the identification of one or more immune response components associated with the one or more computable epitopes of the at least one agent further comprises:
providing one or more predicted courses of an immune response in a host and wherein the one or more predicted courses are responsive to one or more interventions.

12. The method of claim 1, wherein the aiding the identification of one or more immune response components associated with the one or more computable epitopes of the at least one agent includes:
aiding the identification of at least a part of one or more of a T-lymphocyte, a killer T-lymphocyte, a helper T-lymphocyte, a cytotoxic T-lymphocyte, a T-8 lymphocyte, a Cluster of Differentiation (CD) molecule, a CD3 molecule, or a CD1 molecule.

13. The method of claim 1, wherein the aiding the identification of one or more immune response components associated with the one or more computable epitopes of the at least one agent includes:
aiding the identification of at least a part of at least one B-lymphocyte.

14. The method of claim 1, wherein the aiding the identification of one or more immune response components associated with the one or more computable epitopes of the at least one agent includes:
aiding the identification of at least one modulator of at least a part of a B-lymphocyte.

15. The method of claim 1, wherein the aiding the identification of one or more immune response components associated with the one or more computable epitopes of the at least one agent includes:
aiding the identification of one or more of a modulator of at least a part of at least one of a T-lymphocyte, a killer T-lymphocyte, a helper T-lymphocyte, a cytotoxic T-lymphocyte, a T-8 lymphocyte, a Cluster of Differentiation (CD) molecule, a CD3 molecule, or a CD1 molecule.

16. The method of claim 1, wherein the aiding the identification of one or more immune response components associated with the one or more computable epitopes of the at least one agent includes:
aiding the identification of at least a part of one or more of a humanized antibody.

17. The method of claim 1, wherein the aiding the identification of one or more immune response components associated with the one or more computable epitopes of the at least one agent includes:
aiding the identification of one or more of a modulator of at least a part of at least one of a humanized antibody.

18. The method of claim 1, wherein the aiding the identification of one or more immune response components associated with the one or more computable epitopes of the at least one agent includes:
aiding the identification of one or more immune response components directed to at least one of a glycoprotein, and/or a receptor ligand.

19. The method of claim 1, wherein the aiding the identification of one or more immune response components associated with the one or more computable epitopes of the at least one agent includes:
aiding the identification of at least one modulator of (a) an epitopic shift or (b) an epitopic drift predicted in the at least one agent.

20. The method of claim 19, wherein the at least one modulator of (a) an epitopic shift or (b) an epitopic drift predicted in the at least one agent, further comprises:
aiding the identification of at least one suppressor of mutational alteration of the at least one agent.

21. The method of claim 19, wherein the at least one modulator of (a) an epitopic shift or (b) an epitopic drift predicted in the at least one agent, further comprises:
aiding the identification of at least one interfering nucleic acid or nucleic acid sequence.

22. A method, comprising:
identifying an association of at least a computable portion of one or more agents with at least a part of an immune response;
projecting a pattern of one or more changes relating to the at least a computable portion of the one or more agents; and
selecting one or more immune response components in response to the projecting.

23. The method of claim 22, wherein the selecting one or more immune response components further comprises:
selecting at least a part of one or more of a T-lymphocyte, a killer T-lymphocyte, a helper T-lymphocyte, a cytotoxic T-lymphocyte, a T-8 lymphocyte, a Cluster of Differentiation (CD) molecule, a CD3 molecule, or a CD1 molecule.

24. The method of claim 22, wherein the selecting one or more immune response components further comprises:
selecting one or more modulators of at least a part of at least one of a T-lymphocyte, a killer T-lymphocyte, a helper T-lymphocyte, a cytotoxic T-lymphocyte, a T-8 lymphocyte, a Cluster of Differentiation (CD) molecule, a CD3 molecule, or a CD1 molecule.

25. The method of claim 22, wherein the selecting one or more immune response components further comprises:
selecting at least a part of at least one B-lymphocyte.

26. The method of claim 22, wherein the selecting one or more immune response components further comprises:
selecting one or more modulators of at least a part of at least one B-lymphocyte.

27. The method of claim 22, wherein the selecting one or more immune response components further comprises:
selecting at least a part of one or more humanized antibody.

28. The method of claim 22, wherein the selecting one or more immune response components further comprises:
selecting one or more of a modulator of at least a part of at least one humanized antibody.

29. The method of claim 22, wherein the identifying an association of at least a computable portion of one or more agents with at least a part of an immune response further comprises:
identifying an association of at least a computable portion of at least one of a polyglycopeptide, a polysaccharide, a nucleic acid, or a toxin.

30. A system, comprising:
circuitry for identifying an association of at least a computable portion of one or more agents with at least a part of an immune response;
circuitry for projecting a pattern of one or more changes relating to the at least a computable portion of the one or more agents; and
circuitry for selecting one or more immune response components in response to said circuitry for projecting.

31. The system of claim 30, wherein the circuitry for selecting one or more immune response components further comprises:
circuitry for selecting at least a part of one or more of a T-lymphocyte, a killer T-lymphocyte, a helper T-lymphocyte, a cytotoxic T-lymphocyte, a T-8 lymphocyte, a Cluster of Differentiation (CD) molecule, a CD3 molecule, or a CD1 molecule.

32. The system of claim 30, wherein the circuitry for selecting one or more immune response components further comprises:
circuitry for selecting one or more modulators of at least a part of at least one of a T-lymphocyte, a killer T-lymphocyte, a helper T-lymphocyte, a cytotoxic T-lymphocyte, a T-8 lymphocyte, a Cluster of Differentiation (CD) molecule, a CD3 molecule, or a CD1 molecule.

33. The system of claim 30, wherein the circuitry for selecting one or more immune response components further comprises:
circuitry for selecting at least a part of at least one B-lymphocyte.

34. The system of claim 30, wherein the circuitry for selecting one or more immune response components further comprises:
circuitry for selecting one or more modulators of at least a part of at least one B-lymphocyte.

35. The system of claim 30, wherein the circuitry for selecting one or more immune response components further comprises:
circuitry for selecting at least a part of one or more humanized antibody.

36. The system of claim 30, wherein the circuitry for selecting one or more immune response components further comprises:
circuitry for selecting one or more modulators of at least a part of at least one humanized antibody.

37. The system of claim 30, wherein the circuitry for identifying an association of at least a computable portion of one or more agents with at least a part of an immune response further comprises:
circuitry for identifying an association of at least a portion of at least one of a polyglycopeptide, a polysaccharide, a nucleic acid, or a toxin.

38. (canceled)

39. (canceled)

40. A method, comprising:
identifying an association of at least one computable epitope of one or more agents with at least a part of an immune response;
projecting a pattern of one or more changes relating to the at least one computable epitope of the one or more agents; and
selecting one or more immune response components in response to the projecting.

41. The method of claim 40, wherein the selecting one or more immune response components further comprises:
selecting at least a part of one or more of a T-lymphocyte, a killer T-lymphocyte, a helper T-lymphocyte, a cytotoxic T-lymphocyte, a T-8 lymphocyte, a Cluster of Differentiation (CD) molecule, a CD3 molecule, or a CD1 molecule.

42. The method of claim 40, wherein the selecting one or more immune response components further comprises:
selecting one or more modulators of at least a part of at least one of a T-lymphocyte, a killer T-lymphocyte, a helper T-lymphocyte, a cytotoxic T-lymphocyte, a T-8 lymphocyte, a Cluster of Differentiation (CD) molecule, a CD3 molecule, or a CD1 molecule.

43. The method of claim 40, wherein the selecting one or more immune response components further comprises:
selecting at least a part of at least one B-lymphocyte.

44. The method of claim 40, wherein the selecting one or more immune response components further comprises:
selecting one or more modulators of at least a part of at least one B-lymphocyte.

45. The method of claim 40, wherein the selecting one or more immune response components further comprises:
selecting at least a part of one or more humanized antibody.

46. The method of claim 40, wherein the selecting one or more immune response components further comprises:
selecting one or more modulators of at least a part of at least one humanized antibody.

47. The method of claim 40, wherein the identifying an association of at least one computable epitope of one or more agents with a part of an immune response further comprises:
identifying an association of at least one computable epitope of at least one of a polyglycopeptide, a polysaccharide, a nucleic acid, or a toxin.

48. (canceled)

49. (canceled)

50. (canceled)

51. (canceled)

52. (canceled)

53. (canceled)

54. (canceled)
